(19) United States
(12) Patent Application Publication
(10) Pub. No.: US 2010/0222307 A1
(43) Pub. Date: Sep. 2, 2010

(54) VITAMIN D AND VITAMIN D ANALOGS OR DERIVATIVES AS NEW ANTI-HYPERTENSIVE AGENTS

(75) Inventors: Yanchun Li, Chicago, IL (US); Milan Uskokovic, Upper Montclair, NJ (US)

Correspondence Address: BARNES & THORNBURG LLP, P.O. BOX 2786, CHICAGO, IL, 60690-2786, US

(73) Assignee: THE UNIVERSITY OF CHICAGO, Chicago, IL (US)

(21) Appl. No.: 12/715,133

(22) Filed: Mar. 1, 2010

Related U.S. Application Data
(62) Division of application No. 10/962,215, filed on Oct. 8, 2004.
Application No. 10/962,215 is a continuation-in-part of application No. 10/865,624, filed on Jun. 10, 2004, now abandoned.
(60) Provisional application No. 60/477,900, filed on Jun. 12, 2003.

(51) Int. Cl. A61K 31/593; A61P 9/12

(52) U.S. Cl. 514/167

(57) ABSTRACT

Methods and compositions to suppress renin expression and blood pressure in mammals are disclosed. Vitamin D and its analogues and derivatives, including Gemini compounds, are negative regulators of renin synthesis and blood pressure. Renin expression and plasma angiotensin II production were increased several fold in vitamin D receptor (VDR) null mice, leading to hypertension, cardiac hypertrophy and increased water intake. Vitamin D or its analogue-mediated regulation of renin expression and blood pressure is independent of calcium metabolism. Vitamin D analogues or derivatives are novel preventive or therapeutic anti-hypertension agents. Assays to identify novel vitamin D analogues or derivatives as anti-hypertensive agents are disclosed.

### CROSS REFERENCE TO RELATED APPLICATIONS

This application is a divisional of copending U.S. application Ser. No. 10/962,215, filed Oct. 8, 2004, which is a continuation in part of U.S. application Ser. No. 10/865,624, filed Jun. 10, 2004, now abandoned, which claims priority from U.S. provisional application No. 60/477,900, filed Jun. 12, 2003, the contents of which applications are incorporated herein by reference in their entireties.

The U.S. Government has rights in the present invention due to partial support of the Digestive Disease Research Center Grant DK42086 and NIH grant DK 59327.

### BACKGROUND OF THE DISCLOSURE

The renin-angiotensin system is involved in blood pressure, electrolyte and volume homeostasis. Inappropriate activation of the renin-angiotensin system may lead to hypertension, which is a risk factor for stroke, myocardial infarction, congestive heart failure, progressive atherosclerosis and renal failure. The mechanisms of renin-angiotensin processes are not well understood.

Renin, a protease synthesized and secreted predominantly by the juxtaglomerular (JG) apparatus in the nephron is a rate-limiting component of the system. Renin cleaves angiotensin (Ang) I from liver-derived angiotensinogen, which is then converted to Ang II by the angiotensin-converting enzyme. Ang II, through binding to its receptors, exerts diverse actions that affect the electrolyte, volume and blood pressure homeostasis. Inappropriate stimulation of the renin-angiotensin system has been associated with hypertension, heart attack and stroke.

Renin-producing granulated cells are mainly located in the afferent glomerular arterioles in the kidney. Renin secretion is regulated by renal perfusion pressure, renal sympathetic nerve activity and tubular sodium load. Renin secretion is stimulated by factors such as prostaglandins, NO and adrenomedullin, and inhibited by other factors, including Ang II (feedback), endothelin, vasopressin and adenosine. Stimulation of renin secretion is often mediated by an increase in intracellular cAMP and is accompanied by increases in renin gene transcription.

Relationships have been suggested between the vitamin D pathways and blood pressure. Vitamin D is a primary regulator of calcium homeostasis. Genetic inactivation of either the vitamin D receptor (VDR), a member of the nuclear receptor superfamily that mediates the action of 1,25-dihydroxyvitamin D_{3[}1,25(OH)₂D₃], or 25-hydroxyvitamin D₃ 1α-hydroxylase, the rate-limiting enzyme for the biosynthesis of 1,25(OH)₂D₃, results in impaired calcium homeostasis, leading to hypocalcemia, secondary hyperparathyroidism and rickets. However, the mechanism underlying the relationship between vitamin D, blood pressure and plasma renin activity is unknown.

### SUMMARY OF THE DISCLOSURE

Vitamin D and vitamin D analogs or derivatives disclosed herein are new anti-hypertensive agents to control renin production and blood pressure. Vitamin D is a negative regulator of renin expression in vivo.

Disruption of the vitamin D signaling pathway leads to a deregulated elevation of renin expression, and an increase in serum vitamin D levels leads to a suppression of renin expression. Vitamin D is an endocrine suppressor for renin biosynthesis. Mutant mice that lack a vitamin D receptor have much higher production of renin and angiotensin II and develop hypertension and cardiac hypertrophy, whereas injection of 1,25-dihydroxyvitamin D3 into normal mice reduces renin synthesis. Vitamin D analogs with less calcemic effect and higher potency than vitamin D are used for suppressing rennin biosynthesis and regulating blood pressure.

A cell culture system for vitamin D analog screening was developed to identify a group of vitamin D analogs, including Gemini compounds that have more potent renin-suppressing activity than 1,25-dihyroxyvitamin D3 were identified. Gemini compounds suppress renin expression, and a few are 10 to 100 times more potent that 1,25 (OH)₂D₃ (FIG. 4, and FIGS. 1 and 2). Some of the vitamin D analogs that exhibit renin suppressing activity are shown in TABLES 1 and 2.

A method of suppressing renin expression in a mammal includes the steps of:
(a) obtaining a pharmaceutical composition that includes a vitamin D analogue or derivative; and
(b) administering the pharmaceutical composition to the mammal.

A method of reducing blood pressure in a mammal includes the steps of:
(a) obtaining a pharmaceutical composition that includes a vitamin D analogue or derivative; and
(b) administering the pharmaceutical composition to the mammal.

The pharmaceutical composition may have an acceptable carrier and other sustained/extended release formulations. More than type of one vitamin D analogue or derivative can also be formulated as a pharmaceutical composition and administered to mammals including humans.

In an aspect, a vitamin D analogue or derivative may include a Gemini compound. The Gemini compound may have two side chains at C20. Exemplary Gemini compounds include vitamin D analogues or derivatives such as (1,25-dihydroxy-21(3-methyl-3-hydroxy-butyl)-cholecalciferol) (#4); (1,25-dihydroxy-21(3-methyl-3-hydroxy-butyl)-19-nor-cholecalciferol) (#9); (1,25-dihydroxy-20R-21(3-hydroxy-3-methylbutyl)-23-yne-26,27-hexafluoro-cholecalciferol) (#10); (1,25-dihydroxy-20S-21(3-hydroxy-3-methylbutyl)-23-yne-26,27-hexafluoro-cholecalciferol) (#11); (1,25-dihydroxy-20R-21(3-hydroxy-3-methylbutyl)-23-yne-26,27-hexafluoro-19-nor-cholecalciferol) (#12); (1,25-dihydroxy-20S-21(3-hydroxy-3-methylbutyl)-23-yne-26,27-hexafluoro-19-nor-cholecalciferol) (#13); (1,25-dihydroxy-21(2R,3-hydroxy-3-methylbutyl)-20R-cholecalciferol) (#17); and (1,25-dihydroxy-21(2R,3-hydroxy-3-methylbutyl)-20S-cholecalciferol) (#18).

A method of reducing blood pressure in a mammal without inducing hypercalcemia includes the steps of:
(a) obtaining a pharmaceutical composition that includes vitamin D or a vitamin D analogue or derivative that does not induce hypercalcemia; and
(b) administering the pharmaceutical composition to the mammal.

An assay for screening vitamin D analogues or derivatives including Gemini compounds to suppress renin expression in a mammal comprising:
(a) treating (or exposing or adding) a cell culture with vitamin D analogues or derivatives including Gemini compounds;
(b) comparing renin expression in the cell culture treated with the vitamin D analogues or derivatives including Gemini compounds to a control cell culture; and
(c) determining if the vitamin D analogues or derivatives including Gemini compounds suppress renin expression.

An assay for screening vitamin D analogues or derivatives including Gemini compounds to regulate blood pressure in a mammal includes the steps of:
(a) treating a cell culture with vitamin D analogues or derivatives including Gemini compounds;
(b) comparing renin expression in the cell culture treated with the vitamin D analogues or derivatives including Gemini compounds to a control cell culture; and
(c) determining that the vitamin D analogues or derivatives including Gemini compounds regulate blood pressure if renin expression is suppressed.

In an aspect, the cell culture expresses vitamin D receptor (VDR) and renin.

### BRIEF DESCRIPTION OF THE DRAWINGS

FIG. 1 shows an evaluation of the activity of vitamin D analogues to suppress renin expression. Vitamin D analogues listed in TABLE 1 were used to treat As4.1-hVDR cells at 10⁻¹⁰, 10⁻⁹ and 10⁻⁸ M. Renin mRNA was analyzed by Northern blot. 36B4 is a control mRNA and represents a mouse acidic ribosomal phosphoprotein.

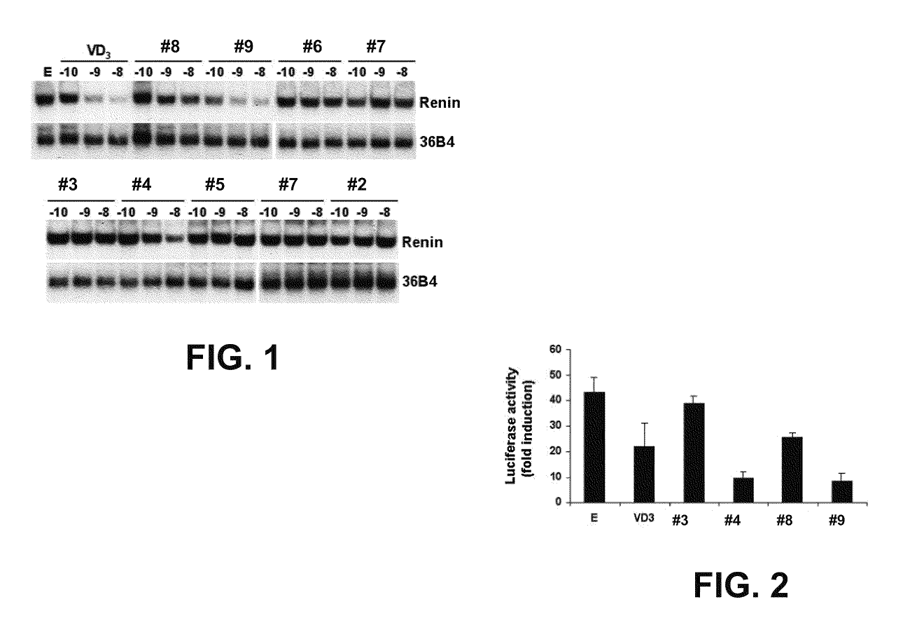

FIG. 2 shows an evaluation of vitamin D analogues by luciferase reporter assays, confirming the super-potency of the Gemini analogues (#4) and (#9) in suppressing the renin promoter activity. As4.1-hVDR cells were transfected with pGL-4.1 kb-Luc, and treated with ethanol (E), 10⁻⁸ M of 1,25(OH)₂D₃ (VD3) and different analogues as indicated.

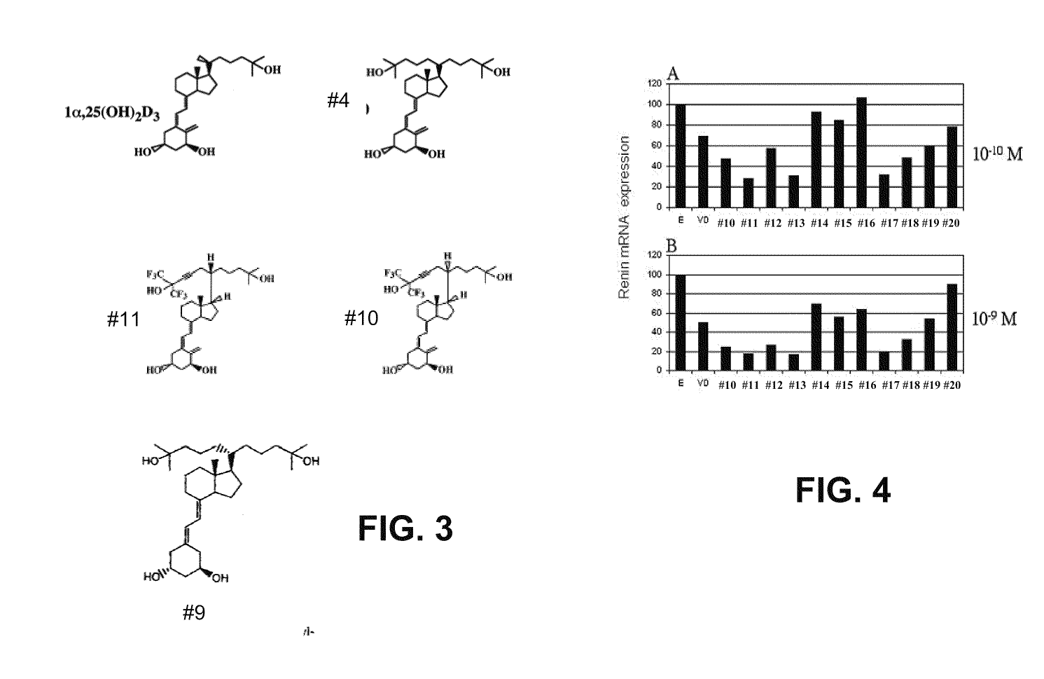

FIG. 3 shows chemical structures of 1,25(OH)₂D₃ and Gemini analogues. All the Gemini compounds listed in TABLES 1 and 2 are derivatives of the two-side chain structure.

FIG. 4 is an histogram of evaluation of Gemini analogues by Northern blot analysis. The 11 Gemini compounds listed in TABLE 2 (indicated) were used to treat As4.1hVDR cells at 10⁻¹⁰, 10⁻⁹, and 10⁻⁸ M for 24 hours. Renin mRNA was quantitated by Northern blots. The quantitative data obtained at 10⁻¹⁰ and 10⁻⁹ M are shown in A and B. The # of each compound corresponds to the number shown in TABLE 2. E, ethanol-treated control, VD, 1,25(OH)₂D₃.

FIG. 5 shows the effects of VDR inactivation on renin expression and plasma Ang II production. A. Renin mRNA expression in the kidney. Kidney total RNAs were isolated from wild type (+/+) and VDR^{-/-} (−/−) mice and analyzed by Northern blot. The same membrane was sequentially hybridized with mouse renin and 36B4 cDNA probes. Each lane represents an individual animal. B. Quantitative results of the Northern blot analyses shown in A. Values represent the ratio of renin mRNA to 36B4 mRNA. *, P<0.001 vs. +/+ mice. C. Immunohistochemical staining of the kidney cortex from wildtype (+/+) and VDR^{-/-} (−/−) mice with anti-renin antiserum. Arrows indicate the afferent glomerular arterioles in the juxtaglomerular region. Scale bar, 25 μm. D. Plasma Ang II concentrations in wildtype (+/+) and VDR^{-/-} (−/−) mice. *, P<0.001 vs. +/+ mice, n=15 in each group. E. Liver angiotensinogen mRNA expression in wildtype (+/+) and VDR^{-/-} (−/−) mice determined by northern blot. The membrane was sequentially hybridized with mouse angiotensinogen and 36B4 cDNA probes. Each lane represents an individual mouse.

FIG. 6 shows the effects of VDR inactivation on blood pressure and heart weight/body weight ratio. A. Systolic and diastolic blood pressures of wildtype (open bar) and VDR^{-/-} (closed bar) mice. *, P<0.01 vs. corresponding +/+ mice. n=9 for +/+ mice; n=8 for −/− mice. B. Ratio of heart weight to body weight of wildtype (+/+) and VDR^{-/-} (−/−) mice. *, P<0.05 vs. +/+ mice; n=9 in each genotype. C. Mean blood pressure (BP) of wildtype (open bar) and VDR^{-/-} (closed bar) mice untreated or treated with captopril for 5 days. *, P<0.05 vs. corresponding untreated +/+ mice. n=4 in each genotype in each group.

FIG. 7 shows the effects of high sodium load and volume depletion on renin mRNA expression and plasma Ang II production in wildtype (+/+) and VDR^{-/-} (−/−) mice. A. Northern blot analysis of renal renin mRNA from mice treated with the normal rodent diet supplemented with 8% NaCl for different days as indicated. Each lane represents an individual mouse. control, untreated. B. Plasma Ang II concentrations in the 8% NaCl diet-treated animals. open bar, +/+ mice; closed bar, −/− mice; *, P<0.01 vs. corresponding +/+ mice at the same time point; **, P<0.05 vs. untreated control +/+ mice; n=3 in each genotype at each time point. C. Northern blot analysis of renal renin mRNA expression in mice dehydrated for 24 hrs (24 hr). Each lane represents an individual mouse. control, untreated. D. Plasma Ang II levels in untreated control and dehydrated (24 hr) mice. open bar, +/+ mice; closed bar, −/− mice; *, P<0.01 vs. corresponding +/+ mice; **P<0.01 vs. untreated control +/+ mice; n=3 in each genotype in each group.

FIG. 8 shows the elevation of renin expression in strontium-treated wildtype mice. Two-month old wildtype mice were fed the normal diet supplemented with 2.5% strontium chloride for 7 weeks before sacrifice. A. Northern blot analysis of renin mRNA expression in the kidney from non-treated and strontium-treated wildtype mice. Each lane represents an individual animal. B. Quantitative results of the northern analysis. C. Blood ionized calcium concentration determined at the end of the treatment (n=5 in each group). In both b and c, NT, non-treated; STR, strontium-treated, *, P<0.01 vs. NT value.

FIG. 9 shows that 1,25-dihydroxyvitamin D₃ suppresses renin expression in wildtype mice. A. Wildtype mice (3 month old) were injected intraperitoneally with 2 or 5 doses of 30 pmole of 1,25(OH)₂D₃ (VD) dissolved in propylene glycol or vehicle (V). The 2 doses were given in two consecutive days at 9 am. The 5 doses were given in three consecutive days at 9 am and 7 pm for the first two days, and at 9 am on the third day. Total renal RNA was isolated 6 hr after the last injection. Renin, calbindin-D9k (CaBP-D9k) and 36B4 mRNA levels were determined by Northern blot analysis. B. Quantitation of renin mRNA levels. Closed bar, vehicle treatment; Open bar, 1,25(OH)₂D₃ treatment. n≧3 in each group. *, P<0.05 vs. vehicle treatment.

FIG. 10 shows that renin up-regulation is independent of the calcium status. A. Blood ionized calcium levels in wildtype (open bar) and VDR^{-/-} (closed bar) mice at 20 days of age (20 d), 3 months of age (3 m) or treated with the HCa-Lac diet for 5 weeks (5 wCa). *, P<0.01 vs. corresponding +/+ value. n≧5 in each group. B. Serum intact PTH (iPTH) concentrations of 20 d, 3 m and 5 wCa wildtype (open bar) and VDR^{-/-} (closed bar) mice. *, P<0.01 vs. corresponding +/+ mice; n≧5 in each group. Note the open bars are barely visible on this scale. C. Northern blot analysis of renal renin mRNA from 20-day old wildtype (+/+) and VDR^{-/-} (−/−) mice. D. Quantitative data of the northern blot analyses from 20-day old mice. *, P<0.001 vs. +/+ mice. E. Northern blot analysis of renin mRNA expression in the kidney of wildtype (+/+) and VDR^{-/-} (−/−) mice treated with the HCa-Lac diet for 5 weeks. F. Quantitative result of the northern blot analysis of the 5 wCa mice. *, P<0.001 vs. +/+ mice. G. Plasma Ang II concentrations of the 5 wCa mice. *, P<0.01 vs. +/+ mice; n=5 in each genotype.

FIG. 11 shows suppression of renin mRNA expression by 1,25(OH)₂D₃ in As4.1 cells. A. As4.1 cells were transiently transfected with (+) or without (−) pcDNA-hVDR plasmid containing the full-length human VDR cDNA and then treated with 5×10⁻⁸ M of 1,25(OH)₂D₃ (+) or ethanol (−) for 24 hrs. Total cellular RNA was isolated and analyzed by northern blot with renin and 36B4 cDNA probes. B. Quantitative results of northern blot analyses obtained from three independent experiments. C, ethanol-treated; VD, 1,25(OH)₂D₃-treated; T, transfected with pcDNA-hVDR and treated with ethanol; T/VD, transfected with pcDNA-hVDR and treated with 1,25(OH)₂D₃. *, P<0.001 vs. C, VD or T value.

FIG. 12 shows that 1,25(OH)₂D₃ suppresses renin gene transcription. A. Expression of hVDR mRNA in stable As4.1 clones. P, parental As4.1 cells; #57, As4.1 clone 57 stably transfected with pcDNA-hVDR; V, As4.1 clone stably transfected with the empty vector pcDNA3.1. B. Renin mRNA expression in As4.1 clone V (Vector) and #57 treated with ethanol (E) or different doses of 1,25(OH)₂D₃ as indicated. c. As4.1-hVDR cells (clone #57) were transfected with pGL3-Control, pGL-4.1 kb or pGL-117 bp luciferase reporter plasmid and then treated with ethanol (closed bar) or 10⁻⁸ M of 1,25(OH)₂D₃ (open bar). Luciferase activity was determined 48 hr after transfection. Similar results were obtained in other stable clones.

FIG. 13 shows renin expression in Gcm2 null mice. A. Northern blot of renin expression; B, quantification of renin expression; C. Calcium levels in the Gcm2 null mice.

FIG. 14 shows the outline of the interaction between the vitamin D endocrine system and the RAS. Under normal physiological conditions 1,25(OH)₂D₃ functions as an endocrine suppressor to maintain the homeostasis of renin production. Renin is also feedback suppressed by Ang II. The mechanism of PTH regulation of renin is unclear. Other studies suggest that vitamin D and PTH may also directly affect the cardiovascular functions (dashed lines).

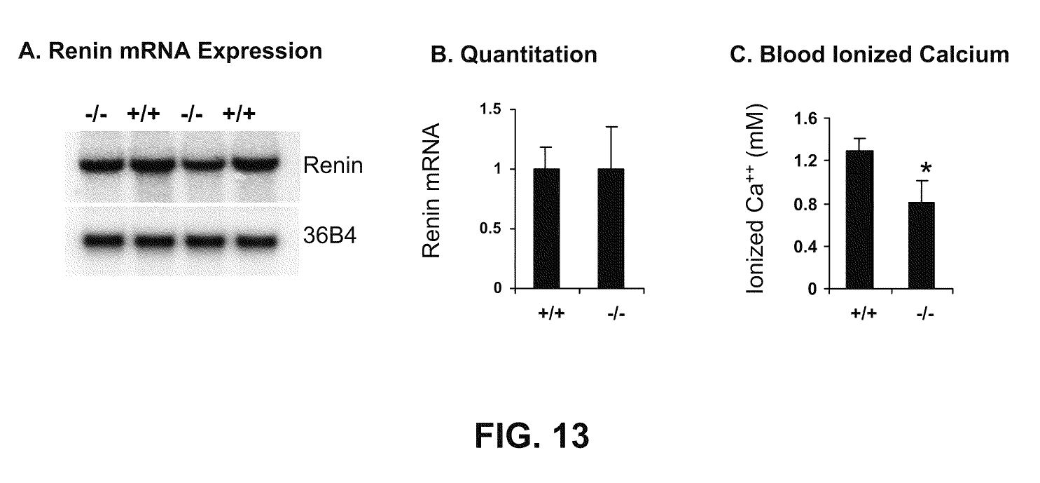

FIG. 15 shows that (#9) reduces renin expression in vivo. Mice were injected i.p. with compound #9 (at 12 μg/kg body weight) or vehicle for 7 days. Kidney RNAs were isolated and renin RNA levels were quantified by Northern blotting. n≧4; p<0.05.

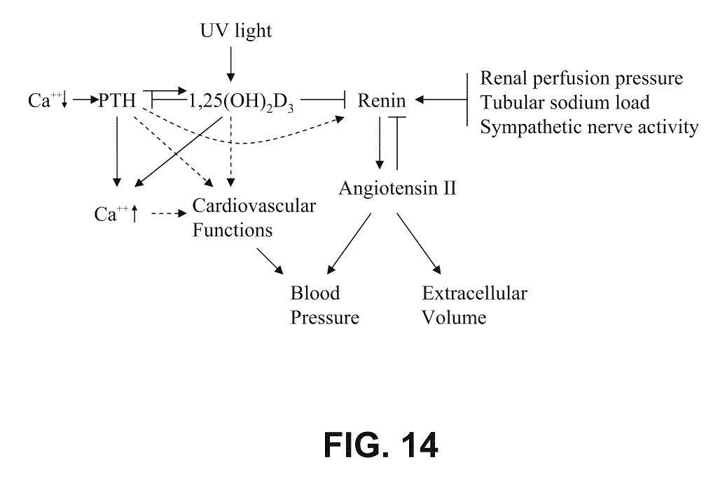

FIG. 16 shows that a Gemini compound inhibits renin expression in normal mice without inducing significant hypercalcemia. (A) Blood ionized calcium levels. Normal mice (n=5) were treated with vehicle (control), 1,25(OH)₂D3 (VD3) or compound #9 at a daily dose of 1.5 μg/kg body weight for one week. Arrow indicates that 2 out of 5 VD3-treated mice died at day 7. (B) Renin mRNA levels in the kidneys of treated mice, as determined by quantitative Northern blots. *, P<0.01.

FIG. 17 shows that a Gemini compound at varying doses can efficiently inhibit renin production in normal mice. Normal mice (n=4 to 6) were treated with compound #9 for one week at a daily dose of 5 μg/kg body weight (A) or 12 μg/kg body weight (B), and renin mRNA expression in the kidneys were determined by Northern blots after the treatment. As a control for vitamin D action, Calbindin-D9k stimulation was measured. Quantitative data were presented below the Northern blot gels. A 54% and 76% reduction in renin mRNA levels after the treatment with the vitamin D analog was observed. *, P<0.001.

FIG. 18 shows renin mRNA expression in wild-type (+/+) and VDR(−/−) mice treated with losartan or captopril. Total kidney RNAs were separated on agarose gels (20 μg/lane). Membranes were hybridized sequentially with 32P-labeled renin and 36B4 cDNA probes. The relative amount of renin mRNA was quantitated after being normalized with 36B4 mRNA levels. (A) Representative Northern blot for renin mRNA levels in untreated and losartan-treated mice. (B) Quantitative results from Northern blots in (A). (C) Representative Northern blot for renin mRNA levels in untreated and captopril-treated mice. (D) Quantitative results from Northern blots in (C). open bar, +/+ mice; filled bar, −/− mice. *, P<0.001 vs. corresponding +/+ values; **, P<0.001 vs. corresponding untreated values of the same genotype.

FIG. 19 shows that treatment with captopril reduces cardiac hypertrophy and ANP up-regulation in VDR(−/−) mice. (A) Heart to body weight ratio of VDR(+/+) and VDR(−/−) mice untreated or treated with captopril for two weeks. *, P<0.001 vs. VDR(+/+) mice; n≧8. (B) Cardiac ANP mRNA levels in VDR(+/+) and VDR(−/−) mice untreated or treated with captopril for two weeks, quantified by Northern blot analysis. *, P<0.05 vs. VDR(+/+) mice, n=4.

FIG. 20 shows the expression of the cardiac renin-angiotensin system in VDR(+/+) and VDR(−/−) mice. Total RNAs were isolated from hearts of VDR(+/+) and VDR(−/−) mice untreated or treated with captopril for two weeks, and the mRNA levels of cardiac renin (A), angiotensinogen (AGT) (B), and AT-1a receptor (C) were quantified by real time RT-PCR. The relative mRNA level of each gene was normalized to GAPDH. *, P<0.01 as compared with corresponding +/+ control; **, P<0.001 as compared with untreated control of the same genotype. n=4 or 5.

### DETAILED DESCRIPTION

Vitamin D and its analogs and derivatives including Gemini compounds suppress renin expression and regulate hypertension in mammals. Vitamin D, and its analogs and derivatives including Gemini compounds are novel anti-hypertensive agents.

A physiological function of the renin-angiotensin system is to maintain vascular resistance and extracellular fluid volume homeostasis, accomplished by the regulatory actions of Ang II on the peripheral vasculature, heart, central nerve system, kidney and adrenal glands. The renin-angiotensin cascade, a rate-limiting component of renin secretion and production, is mostly stimulated by volume or salt depletion, reduction in renal vascular perfusion pressure and sympathetic nerve activity.

VDR null mice have a sustained elevation of renin expression while still maintaining a normal level of blood electrolytes. The augmentation of renin synthesis leads to increased plasma Ang II production from angiotensinogen, which drives VDR null mice to increase water intake and intestinal salt absorption, because Ang II is a very potent thirst-inducing agent that acts on the central nervous system, as well as a potent stimulator of intestinal sodium absorption. The mutant mice have to excrete more urine and salt to maintain volume and electrolyte homeostasis. As a potent vasoconstrictor, Ang II augmentation also leads to the development of hypertension and cardiac hypertrophy in VDR null mice. Thus, a new steady state of the renin-angiotensin system is established in VDR null mice, in which the basal renin expression is higher but still responds appropriately to the same tubular salt load and volume stimuli as in the normal state. It is believed that the up-regulation of renin expression is a primary defect in VDR null mice.

### EXAMPLES

### Example 1

### VDR Null Mice Maintain a High Level of Renin Expression

Renin expression in VDR null mice, which reacts to high salt load or dehydration indicates that the mechanism underlying the sustained renin elevation is independent of the pathways activated by tubular salt load or volume depletion. The involvement of COX-2 implicated in mediating macula densa-mediating renin release, in renin elevation in VDR null mice is unlikely, because the same low COX-2 protein level was observed in the kidney of both VDR null and wildtype mice. Because adult VDR null mice develop hypocalcemia and secondary hyperparathyroidism, the up-regulation of renin expression could be due to VDR inactivation per se, hypocalcemia and/or high PTH. However, vitamin D regulation of renin gene expression is direct and independent of the calcium status because: (1) Pre-weaned VDR null mice that have not yet developed hypocalcemia already show an elevated renin expression; (2) When the blood ionized calcium of adult VDR null mice is normalized by the HCa-Lac diet, their renin expression and Ang II level are still elevated; (3) Conversely, Gcm2 null mice, which are as hypocalcemic as VDR null mice, do not manifest elevated renin expression; (4) In wildtype mice, reduction of 1,25(OH)₂D₃ biosynthesis also results in elevated renin expression, whereas injection of 1,25(OH)₂D₃ leads to reduced renin expression; and (5) 1,25(OH)₂D₃ directly suppresses renin gene transcription in As4.1 cells by a VDR-mediated mechanism. Vitamin D is a potent negative endocrine regulator of renin expression in vivo.

Secondary hyperparathyroidism may also contribute to the renin up-regulation in VDR null mice, because the serum PTH level in the normocalcemic pre-weaned or HCa-Lac diet-treated VDR null mice is still significantly higher than that of the wildtype mice (even though it is much lower than that of the untreated adult VDR null mice). PTH may indirectly regulate renin expression in vivo.

### Example 2

### 1,25(OH)₂D₃ Exerts its Actions by Binding to the VDR

In most cases where 1,25(OH)₂D₃ acts as a positive regulator, the liganded VDR heterodimerizes with the RXR and binds to specific DNA sequences (VDRE) in the promoter of target genes to regulate gene expression. 1,25(OH)₂D₃ also acts as a negative regulator. VDR-mediated transcriptional repression 1,25(OH)₂D₃ appears to suppress renin gene expression through a cis-DNA element(s) in the renin gene promoter.

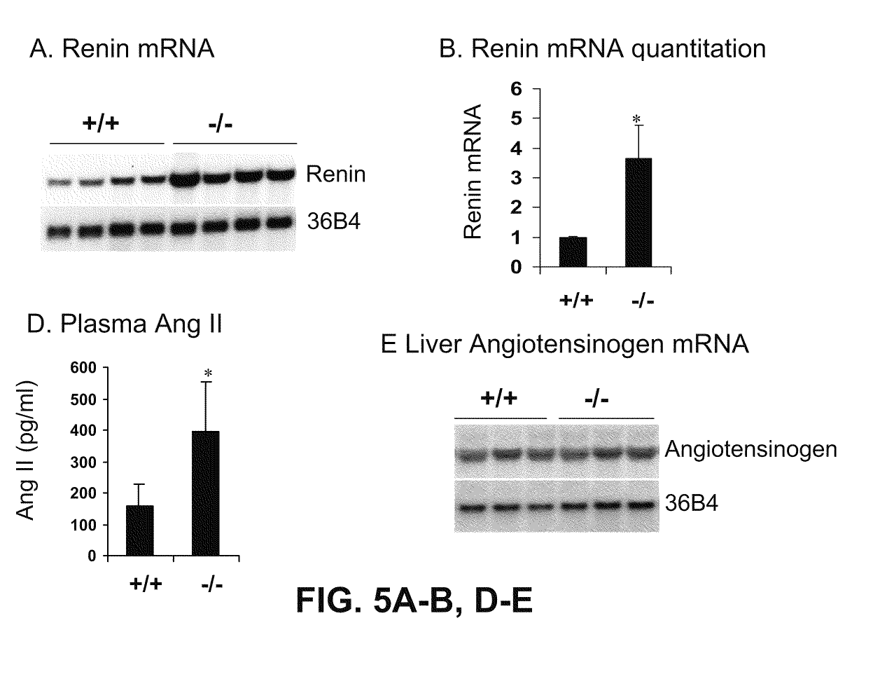

FIG. 3 shows the structure of 1,25-dihydroxyvitamin D3, the most active, hormonal form of vitamin D, and the basic structure of the Gemini compounds and some Gemini analogues. As used herein, vitamin D analogs or derivatives include all structures that resemble vitamin D and include Gemini compounds and their analogues or derivatives.

### Example 3

### Screening Assay for Identifying Gemini Compounds in Suppressing Renin Expression

The results of vitamin D analog screening using the cell culture system are summarized in TABLE 1. Of the 9 compounds, two compounds (as indicated by an *) are found as active as, or more active than, 1,25-dihydroxyvitamin D3. Both the active compounds are Gemini compounds. The results of more vitamin D analog screening are summarized in TABLE 2. At least 8 compounds either were as active or more potent than 1,25-dihydroxyvitamin D3 in suppressing renin gene expression. These compounds are suitable for animal testing. Some of these active compounds (e.g. 10, 11, 12, 13, 17, 18) were at least 10- to 100-fold more potent than 1-25-dihydroxyvitamin D3, whereas they are known to have less side effects than 1,25-dihydroxyvitamin D3, rendering them candidates for further testing.

These results demonstrated the feasibility of using the screening system of the present invention to screen potentially a large number of vitamin D analog compounds to identify the most promising ones for animal and human trials. The renin-angiotensin system (RAS) is a direct target for vitamin D to regulate blood pressure. RAS as a vitamin D target plays a role in regulating hypertension. RAS can also be targeted by vitamin D analogues or derivatives including Gemini compounds and their analogues and derivatives.

### Example 4

### Renin Expression and Plasma Ang II Production are Elevated in VDR Null Mice

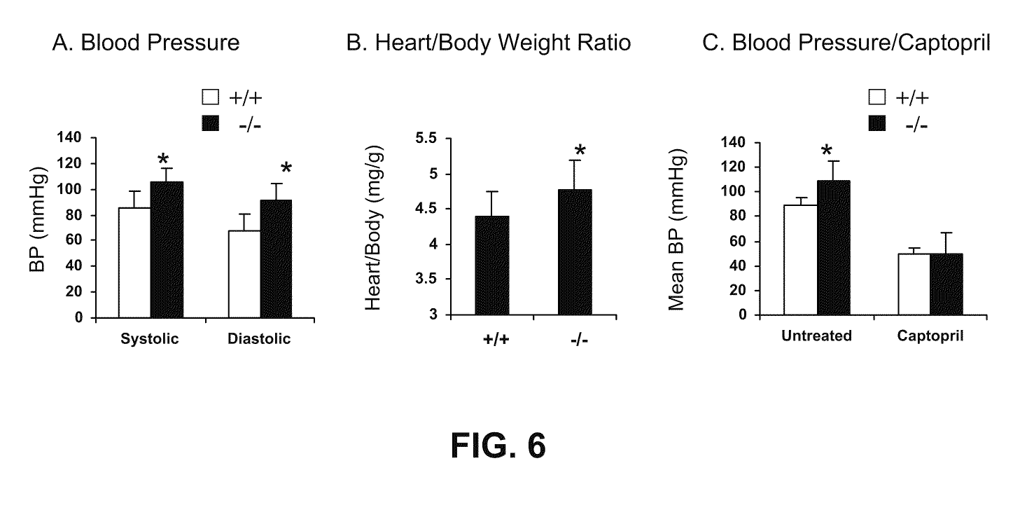

Renin expression was increased in VDR (vitamin D receptor) null mutant mice because of the disruption of the vitamin D signaling pathway. Quantitative northern blot analysis, showed that the renin mRNA level in the kidney of adult VDR^{-/-} mice was more than 3-fold higher than that of wildtype littermates (FIG. 5A-B). Immunohistochemical analysis of the renal cortex with an anti-renin antibody confirmed an dramatic increase in renin immunoreactivity in the afferent glomerular arterioles of the JG region in VDR^{-/-} mice (FIG. 5C). The plasma Ang II level of VDR^{-/-} mice was also increased more than 2.5-fold as compared with wildtype mice (FIG. 5D). However, the expression of angiotensinogen, the precursor of Ang II, in the liver of VDR^{-/-} mice was the same as wildtype mice (FIG. 5E), suggesting that the increase in plasma Ang II was mainly due to the increase in renin activity. Vitamin D negatively regulates renin expression.

### Example 5

### VDR Null Mice Exhibited Increased Blood Pressure

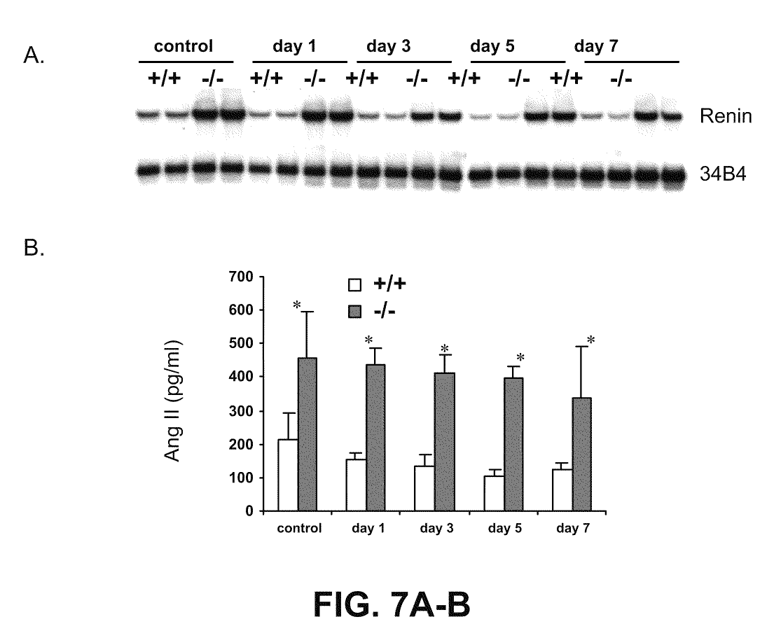

Ang II is a potent vasoconstrictor. The blood pressure of VDR^{-/-} and wildtype mice were compared. Both the systolic and diastolic pressures of VDR^{-/-} mice were significantly higher (>20 mmHg) than those of wildtype littermates (FIG. 6A), indicating that VDR^{-/-} mice are hypertensive. The heart weight to body weight ratios of the mutant mice were also significantly higher (FIG. 6B), suggesting that the adult VDR^{-/-} mice developed cardiac hypertrophy. When the mice were treated with captopril, an angiotensin-converting enzyme inhibitor, the blood pressure of both wildtype and VDR^{-/-} mice was reduced. However, no difference was seen between the blood pressures of the treated wildtype and VDR^{-/-} mice (FIG. 6C). This result demonstrated that the increase in the blood pressure of VDR^{-/-} mice is due to renin and plasma Ang II elevation.

### Example 6

### VDR Null Mice Show Abnormal Drinking Behavior

Ang II is known to be a very potent stimulus for thirst and salt craving as well as an inducer of intestinal water and sodium absorption. Water and food intake as well as blood and urinary electrolyte parameters were measured. As shown in TABLE 3, VDR^{-/-} mice ingested about twice the amount of water as the wildtype littermates, and consequently, excreted approximately twice the amount of urine. The abnormal drinking behavior is not due to diabetes, because the blood glucose and insulin levels of VDR^{-/-} mice were normal (TABLE 4). Food intake of VDR^{-/-} mice was similar to wildtype mice, but they excreted 37% and 19% more Na⁺ and K⁺ in the urine, respectively (TABLE 3), while maintaining a normal concentration of blood Na⁺ and K⁺ (TABLE 4). Thus, VDR^{-/-} mice appeared to have an increase in the intestinal salt absorption due to the Ang II elevation.

### Example 7

### VDR Null Mice Respond to Salt Load or Volume Change

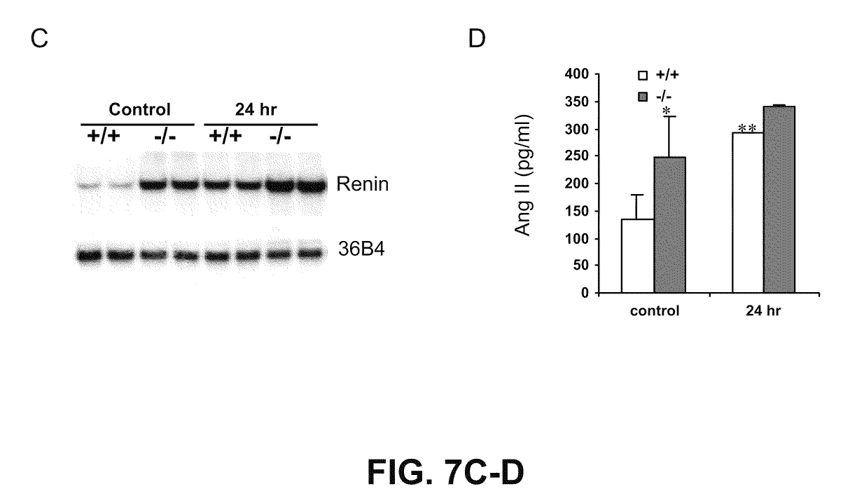

Renin production is very sensitive to changes in tubular salt load or extracellular fluid volume. The effect of high salt diet or dehydration on the expression of renin in VDR^{-/-} and wildtype littermates was investigated. When placed on a normal diet supplemented with 8% NaCl, both VDR^{-/-} and wildtype mice responded by reducing the expression of renin mRNA, but VDR^{-/-} mice still maintained a significantly higher renin mRNA level even after 7 days on the high salt diet (FIG. 7A). Similar changes were seen in the plasma Ang II levels in these animals (FIG. 7B). When the mice were dehydrated for 24 hr, which leads to hypovolemia, they responded by increasing renin mRNA synthesis, but the increase in wildtype mice was more dramatic than in VDR^{-/-} mice (FIG. 7C), indicating that the basal renin production in VDR^{-/-} mice was already near the maximal capacity. The changes of plasma Ang II concentrations in the dehydrated mice were consistent with the changes in the renin expression (FIG. 7D). These observations indicated that, despite a high basal renin synthesis, the regulatory mechanisms activated by tubular salt load changes or volume depletion are still intact in VDR^{-/-} mice. These data also indicate that the elevation of the basal renin expression in VDR^{-/-} mice is through a different mechanism than the physiological inducers.

### Example 8

### Inhibition of 1,25-dihydroxyvitamin D₃ Biosynthesis Leads to Renin Up-Regulation

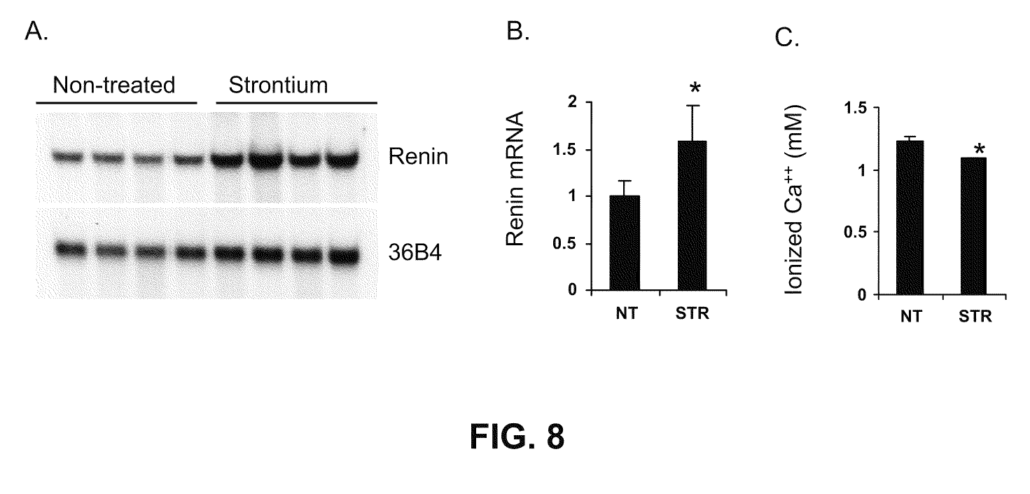

Dietary strontium has been shown to block the biosynthesis of 1,25(OH)₂D₃ and is widely used to render animals vitamin D-deficient. To confirm that the disruption of the vitamin D signaling can lead to renin up-regulation, wildtype mice were treated with strontium. The blood ionized calcium was monitored, instead of the blood 1,25(OH)₂D₃ level, during the treatment because of the extreme difficulty to measure the serum 1,25(OH)₂D₃ concentration in live mice. As shown in FIG. 8, after seven weeks of treatment, the wildtype mice became hypocalcemic (FIG. 8C), indicating that the concentration of 1,25(OH)₂D₃ was already reduced, since 1,25(OH)₂D₃ is required to maintain the calcium homeostasis. The treated mice showed a significant increase in renin mRNA expression (FIG. 8A-B), consistent with the suppressive role of 1,25(OH)₂D₃ in renin expression.

### Example 9

### 1,25-dihydroxyvitamin D₃ Treatment Suppresses Renin Expression in Wildtype Mice

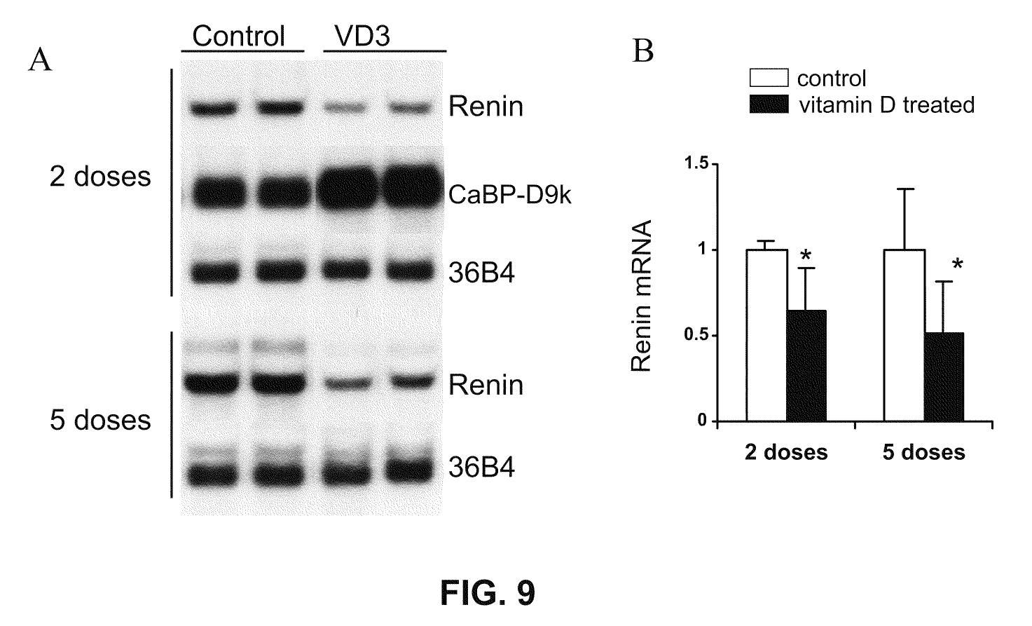

1,25(OH)₂D₃ indeed suppressed renin expression in vivo. Wildtype mice were treated with 1,25(OH)₂D₃ or vehicle and then the renin mRNA level in the kidney was determined. After two doses of 1,25(OH)₂D₃ (30 pmole/dose) in two consecutive days, renal renin expression was decreased by 35%, and after five doses in three days, the expression was decreased by 50% (FIG. 9A-B). As a control, the mRNA of renal calbindin-D9k, a vitamin D target gene, was significantly increased by the 1,25(OH)₂D₃ treatment (FIG. 9A). Thus, the data obtained from VDR^{-/-} mutant mice and from strontium- and 1,25(OH)₂D₃-treated wildtype mice confirm the existence of a negative regulatory interaction between vitamin D and the renin-angiotensin system in vivo.

### Example 10

### Elevation of Renin Expression is Independent of Hypocalcemia

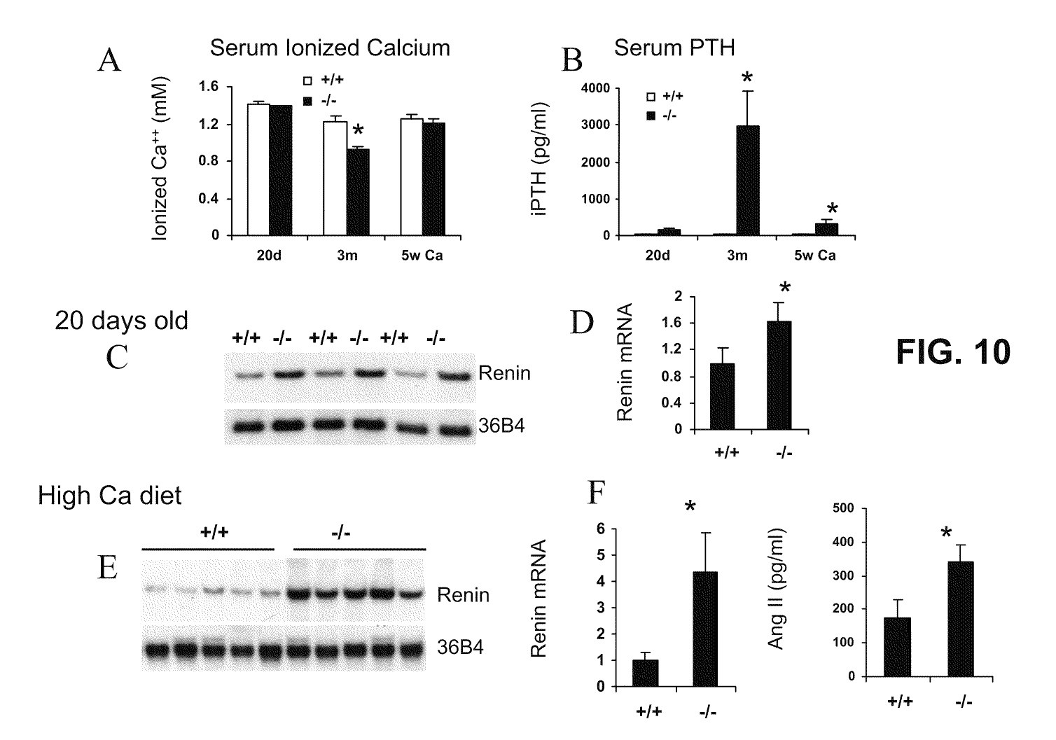

Because vitamin D is a primary regulator of calcium homeostasis, changes in the vitamin D status altered the blood levels of calcium and PTH in animals. Adult VDR^{-/-} mice developed hypocalcemia and secondary hyperparathyroidism. As shown in FIG. 10, their blood ionized calcium level was decreased by 30% and serum PTH concentration increased about 150-fold at 3 months of age (FIG. 10A). Whether the effect of VDR inactivation on renin expression in vivo is direct, or is only secondary to changes in the blood calcium or PTH level was investigated. Because hypocalcemia may reduce the intracellular calcium concentration and cause the renin up-regulation, and high PTH may also stimulate renin secretion, calcium and PTH levels were measured. 20-day old VDR^{-/-} mice that were still normocalcemic (FIG. 10A), but already showed a six-fold increase in the serum PTH level (FIG. 10B), likely due to the lack of the VDR-mediated vitamin D inhibition of PTH biosynthesis were examined. A significant increase in renin expression was seen in these pre-weaned VDR^{-/-} mice (FIG. 10C). Adult VDR^{-/-} mice treated with the HCa-Lac diet that contains 2% calcium, 1.25% phosphorus and 20% lactose were examined. Five weeks of dietary treatment normalized the blood ionized calcium level in VDR^{-/-} mice (FIG. 10A), and reduced the serum PTH concentration of VDR^{-/-} mice to about seven times the wildtype value (FIG. 10B), but had no effects on the concentration of blood electrolytes (TABLE 4). However, renin mRNA and plasma Ang II levels in these normocalcemic adult VDR^{-/-} mice were still significantly elevated (FIG. 10D-E). Similarly, their water intake and urinary excretion were also significantly higher. In addition, renin expression was still elevated in VDR^{-/-} mice whose alopecia was rescued by targeted expression of human VDR in the skin, indicating that the up-regulation of renin expression is not due to alopecia.

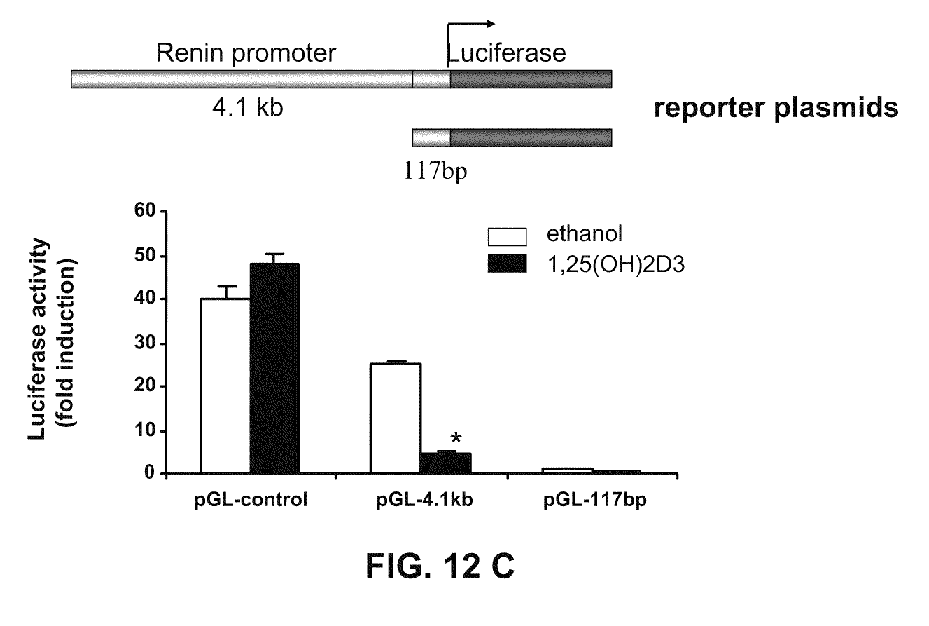

To exclude the possibility that hypocalcemia may increase renin expression, renin expression was examined in Gcm2^{-/-} mice that lack the parathyroid glands (Gcm2 is a master regulatory gene for parathyroid gland development), but have normal circulating PTH (derived from the thymus) and 1,25(OH)₂D₃ concentrations (FIG. 13). Although the blood ionized calcium of Gcm2^{-/-} mice was as low as that of VDR^{-/-} mice, no increase in renin mRNA expression was detected in Gcm2^{-/-} mice (FIG. 13A-C). These data demonstrated that the elevation of renin expression is not due to hypocalcemia, but resulted from VDR inactivation per se and/or hyperparathyroidism.

### Example 11

### Vitamin D Directly Suppresses Renin Expression

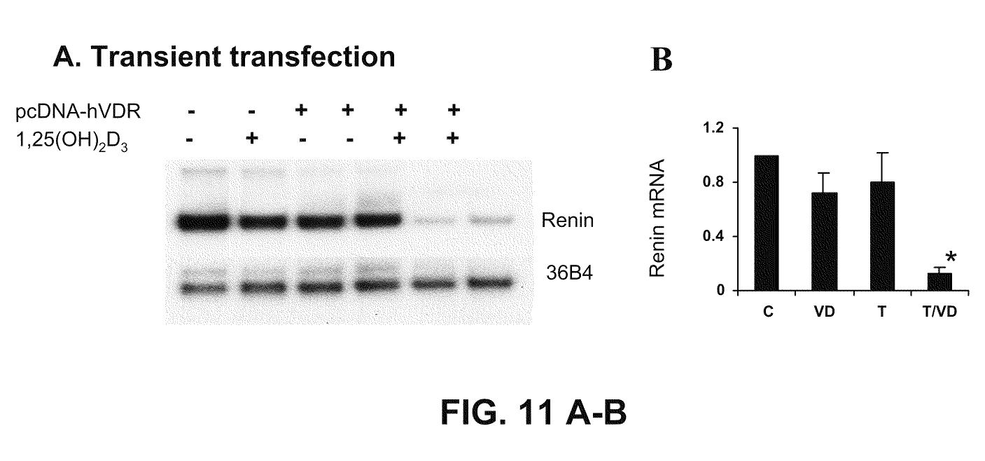

Vitamin D directly suppressed renin gene expression. The effect of 1,25(OH)₂D₃ treatment was examined on renin mRNA expression in As4.1 cells, a JG cell-like cell line that was derived from kidney tumors of SV40 T antigen (Simian Virus 40) transgenic mice and maintains a high level of renin synthesis. Treatment with 5×10⁻⁸ M of 1,25(OH)₂D₃ caused a moderate reduction in renin mRNA expression; however, when the cells were transiently transfected with the pcDNA-hVDR plasmid that contained the full-length coding sequence of human VDR cDNA, the same 1,25(OH)₂D₃ treatment reduced renin mRNA expression by about 90% (FIG. 11). Thus, 1,25(OH)₂D₃ directly suppresses renin expression in a VDR-dependent manner.

### Example 12

### Vitamin D Suppressed Renin Gene Promoter Activity

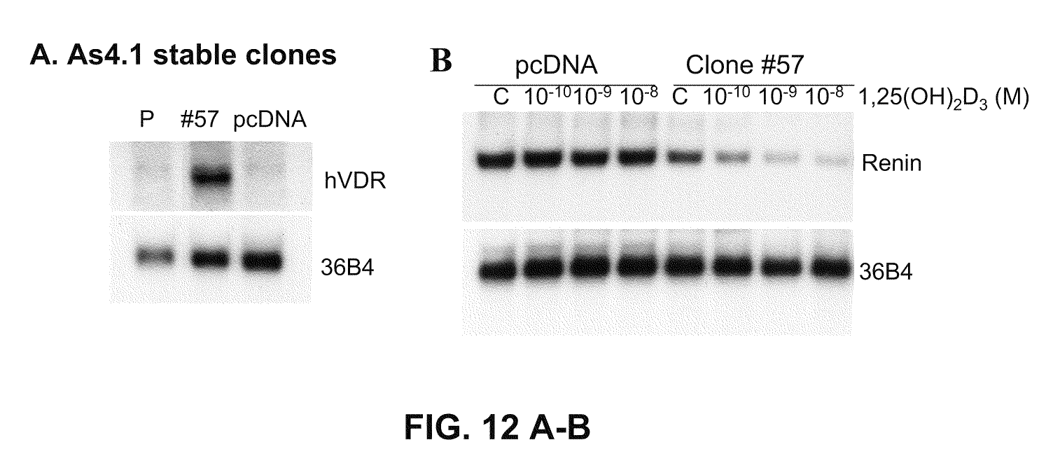

In the As4.1 cells, which have lost expression of some nuclear receptors such as LXR, the VDR mRNA transcript was undetectable by northern blot. As4.1 clones stably transfected with the pcDNA3.1 vector or pcDNA-hVDR were established (FIG. 12A). When the stable clones were treated with 1,25(OH)₂D₃, a dose-dependent suppression of renin expression was seen in As4.1-hVDR cells, but not in As4.1-pcDNA cells (FIG. 12B). The level of renin mRNA was reduced by about 90% in As4.1-hVDR cells treated with 10⁻⁸ M of 1,25(OH)₂D₃. Time-course studies showed that the suppression of renin mRNA was evident after 6 hr of 1,25(OH)₂D₃ treatment. The VDR-mediated suppression was at the transcriptional level. This was determined by measuring the activity of the renin gene promoter was measured in As 4.1-hVDR cells. As shown in FIG. 12C, transfection of the cells with the pGL-4.1 kb reporter plasmid containing the 4.1 kb 5′-flanking sequence of the murine renin gene (Ren-1^{c}) resulted in a 25-fold increase in luciferase activity; treatment of the transfected cells with 1,25(OH)₂D₃ reduced the activity of the 4.1 kb renin gene promoter by more than 80%, but had no effect on the activity of the SV40 promoter in pGL3-Control plasmid. Thus, the suppression of the renin gene promoter by 1,25(OH)₂D₃ is potent and specific. The 117 by 5′-flanking fragment had very low activity. These results demonstrated that 1,25(OH)₂D₃ directly and negatively regulates renin gene transcription through a VDR-mediated mechanism.

### Example 13

### Gemini Compounds Effectively Inhibit Renin Expression in Normal Mice In Vivo without Inducing Significant Hypercalcemia

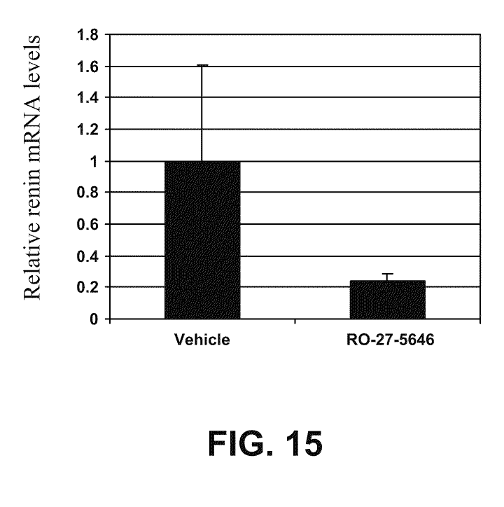

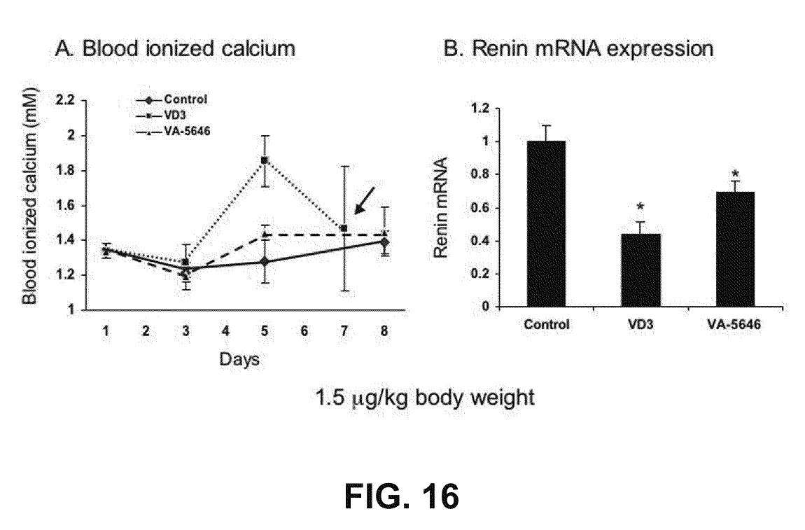

Germini compounds (analogues and derivatives) that show potent activity to suppress renin expression in vitro, need to work in vivo in order for them to be used as therapeutic renin inhibitors or as pharmaceutical compositions. The Gemini compound #9 was tested as a model compound in normal mice. As shown in FIG. 16, when this compound and 1,25(OH)₂D₃ were used to treat mice in parallel, 1,25(OH)₂D₃ induced severe hypercalcemia within four days at a relatively low dose (1.5 μg/kg body weight), resulting in the death of 2 animals out of the 5 mice on day 7 (FIG. 16A). In contrast, the same dose of (#9) only caused a minimal increase in blood calcium level (FIG. 16A). Renin mRNA expression in the kidneys was reduced by these treatments (FIG. 16B). A higher dose of compound #9 (5 μg/kg body weight or 12 μg/kg body weight) was used to treat the mice, which reduced renin expression in the kidneys even more dramatically (FIGS. 17A and B).

Thus, renin expression was significantly reduced in vivo in normal mice treated with compound #9 without a significant increase blood calcium levels. Vitamin D analogues/derivatives including Gemini compounds or their derivatives such as compound #9 can be used as anti-hypertensive agents.

### Example 14

### Effect of Angiotensin II type I Receptor Antagonist and Angiotensin-Converting Enzyme Inhibitor on Vitamin D Receptor Null Mice

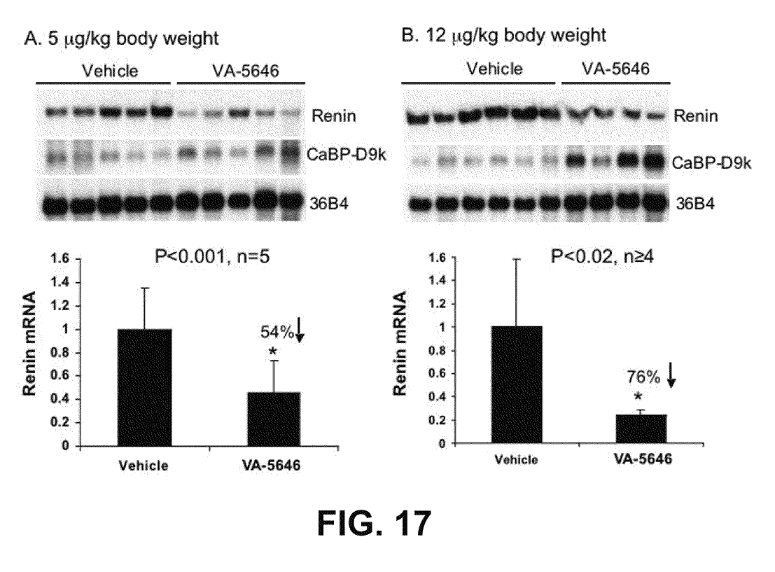

The relationship between RAS activation and the abnormalities in electrolyte and volume homeostasis was performed by analyzing the effects of AT1 receptor antagonist losartan and angiotensin-converting enzyme inhibitor captopril on VDR-null mice. Treatment with losartan or captopril normalized the water intake and urine excretion of VDR-null mice. However, the increase in salt excretion in VDR-null mice was not affected by either drug, indicating that this abnormality is independent of the RAS. Northern blot and immunohistochemical analyses revealed that both drugs caused a drastic stimulation of renin expression in both wild-type and VDR-null mice, but renin expression in the treated VDR-null mice remained much higher than in the treated wild-type mice as shown in FIG. 18, indicating that the angiotensin (Ang) II feedback mechanism remained intact in the mutant mice. VDR(−/−) mice had more than 3-fold increase in renin mRNA expression as compared to wild-type mice (FIG. 18A-D). After losartan or captopril treatment, renin mRNA levels in both wild-type and VDR(−/−) mice were drastically increased (approximately 5- to 8-fold higher than the respective untreated levels). The treated VDR(−/−) mice still expressed 2 to 3 times more renin mRNA transcripts than the treated wild-type mice (FIG. 18A-D). The level of renin protein, as assessed by renin-specific antibody in immunohistochemical staining, was consistent with the mRNA level.

These data support a causative relationship between RAS over-stimulation and the abnormal volume homeostasis in VDR-null mice, and demonstrated that the vitamin D repression of renin expression is independent of the Ang II feedback regulation in vivo. The increase in renin expression seen in the treated animals is due to the disruption of the Ang II feedback regulation caused by the drug treatment, although changes in other physiological parameters (such as perfusion pressure, sympathetic output and/or tubular sodium load) that may also affect renin expression. These results support that the regulatory mechanisms for renin production, including the Ang II feedback regulation, are functionally intact in VDR(−/−) mice. Thus, Ang II feedback repression and vitamin D repression of renin expression are independent negative regulatory pathways to maintain the homeostasis of the RAS.

### Example 15

### Cardiac Hypertrophy in Vitamin D Receptor Knockout (VDRKO) Mice

Cardiac hypertrophy, usually characterized by enlarged left ventricular myocytes, is a common and often lethal complication of arterial hypertension. At the molecular level, cardiac hypertrophy is often accompanied by activation of the so-called fetal gene program in the left ventricle. This program includes the genes encoding atrial natriuretic peptide (ANP), α-skeletal actin, and β-myosin heavy chain. These genes are normally expressed in late fetal and early neonatal heart tissues and are extinguished in adult ventricular myocardium. The increase of ANP is regarded as a cardio-protective response because of the associated natriuretic, anti-hypertrophic, anti-fibrotic and anti-hypertensive activities.

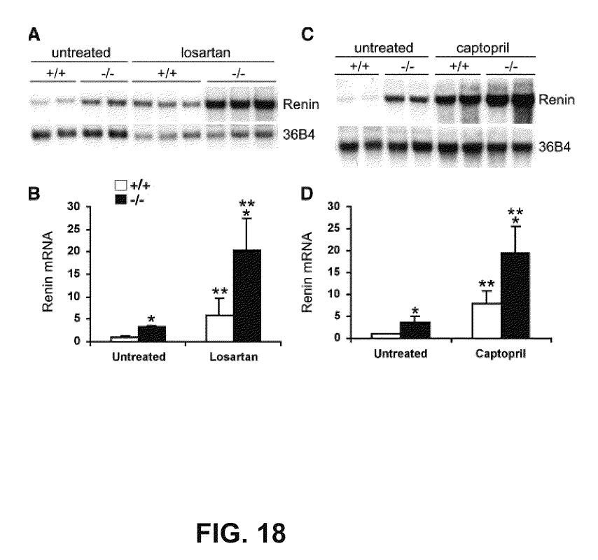

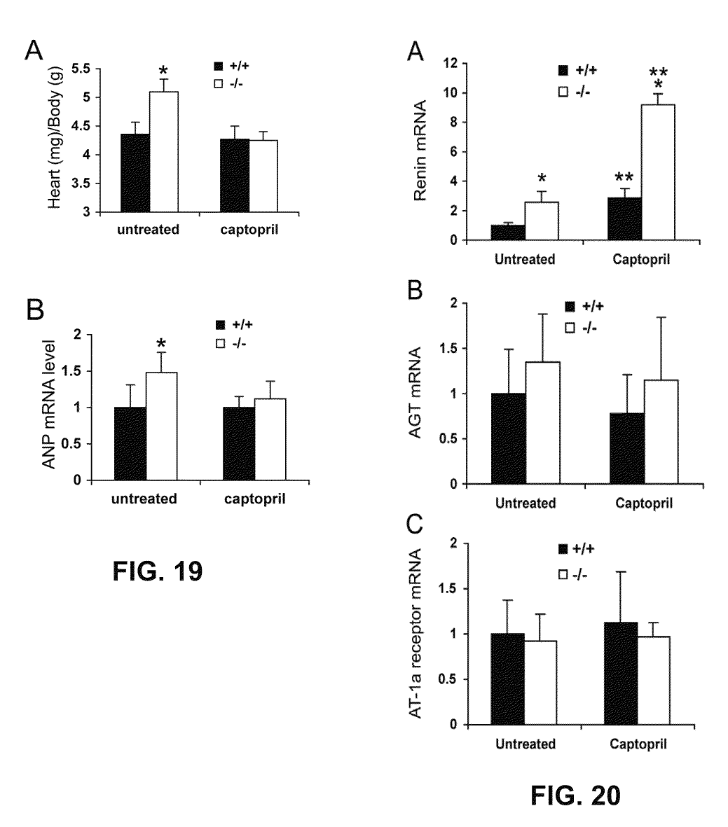

As disclosed herein, 1,25-dihydroxyvitamin D₃ is an endocrine suppressor of renin biosynthesis. Genetic disruption of the vitamin D receptor (VDR) resulted in over-stimulation of the renin-angiotensin system (RAS), leading to high blood pressure and cardiac hypertrophy. Consistent with the higher heart-to-body weight ratio, the size of left ventricular cardiomyocytes in VDR knockout (KO) mice was increased compared to wild-type mice. The levels of atrial natriuretic peptide (ANP) mRNA and circulating ANP were also increased in VDRKO mice. Treating VDRKO mice with captopril reduced cardiac hypertrophy and normalized ANP expression (FIG. 19). The expression of renin, angiotensinogen and AT-1a receptor in the heart was examined by real-time RT-PCR and immunostaining to analyze the role of the cardiac RAS in the development of cardiac hypertrophy. In VDRKO mice, the cardiac renin mRNA level was increased, and this increase was further amplified by captopril treatment (FIG. 20). Intense immunostaining was detected in the left ventricle of captopril-treated WT and VDRKO mice using an anti-renin antibody. Levels of cardiac angiotensinogen and AT-1a receptor mRNAs were unchanged in the mutant mice. The captopril treatment had little effect on the heart to body weight ratio in WT mice, but significantly reduced the heart to body weight ratio in VDR(−/−) mice (FIG. 19A). Similarly, the treatment also reduced ANP mRNA expression in VDR(−/−) mice to the levels detected in WT mice (FIG. 19B). These results indicate that the cardiac hypertrophy and increased ANP gene expression in VDR(−/−) mice are largely a consequence of elevated Ang II production resulting from RAS activation.

Rrenin, AGT and AT-1aR mRNA levels were measured within the hearts of VDR(−/−) mice by real-time RT-PCR. As shown in FIG. 20, the mRNA level of cardiac renin was increased by more than 2.5-fold in VDR(−/−) mice compared to WT mice (FIG. 20A). Interestingly, captopril treatment dramatically induced cardiac renin mRNA expression in both WT and VDR(−/−) mice, and renin mRNA levels in the treated VDR(−/−) mice remained much higher than those seen in the treated WT mice (FIG. 20A).

These data support the notions that the cardiac hypertrophy seen in VDRKO mice is a consequence of activation of both the systemic and cardiac RAS, and that 1,25-dihydroxyvitamin D₃ regulates cardiac functions, at least in part, through the RAS.

TABLE 1
Renin suppressive activity of Vitamin D analogues

| Vitamin D Analogues | Suppressive Activity |
|---|---|
| (#1) | − |
| (#2) | − |
| (#3) | − |
| (#4)* | ++ |
| (#5) | − |
| (#6) | − |
| (#7) | − |
| (#8) | + |
| (#9)* | +++ |

*Indicates Gemini analogues.

TABLE 2
Renin suppressive activity of Gemini Analogues

| Gemini analogue | Relative inhibitory activity* |
|---|---|
| (#4) | ++ |
| (#9) | +++ |
| (#10) | +++ |
| (#11) | ++++ |
| (#12) | +++ |
| (#13) | ++++ |
| (#14) | +/− |
| (#15) | + |
| (#16) | +/− |
| (#17) | +++ |
| (#18) | ++ |
| (#19) | + |
| (#20) | − |
| 1,25-dihydroxyvitamin D₃ | ++ |
| (#21) | |

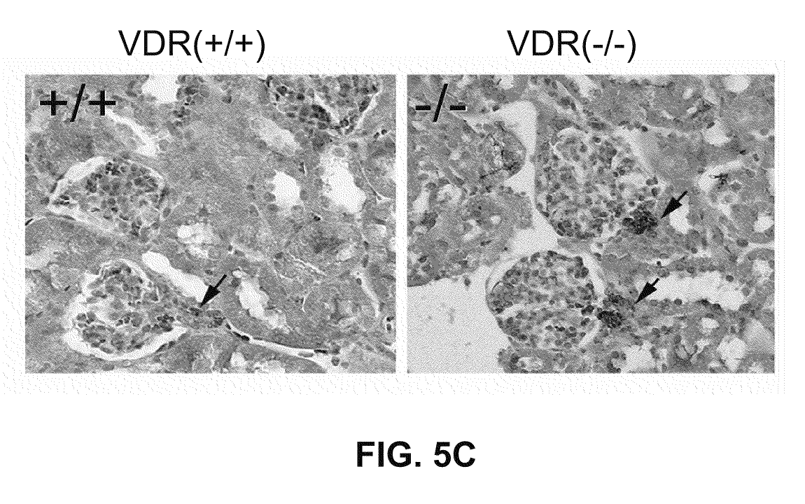

*The inhibitory activity was determined by measuring the renin mRNA level by Northern blot analyses after treating As4.1-hVDR cells with each Gemini compound for 24 hours at 10-8, 10-9 and 10-10 M. The relative activity is based on that of 1,25-dihydroxyvitamin D3, which is arbitrarily set at ++.
#refers to the compound number in FIG. 4.

TABLE 3
Twenty-four hr water and food intake, urinary volume and urinary
electrolyte concentrations in VDR−/− mice compared to wild-type.

| | Wild-type | VDR^{-/-} | P value |
|---|---|---|---|
| Water | 2.7 ± 0.3 (n = 29) | 5.4 ± 0.4 (n = 29) | <0.01 |
| (ml/mouse/day) | | | |
| Food | 138.9 ± 12.2 (n = 29) | 142.9 ± 13.1 (n = 29) | ns |
| (g/kg BW/day) | | | |
| Urine | 1.1 ± 0.4 (n = 9) | 1.8 ± 0.5 (n = 9) | <0.01 |
| (ml/mouse/day) | | | |
| Urinary Na⁺/Cr | 2.9 ± 0.6 (n = 9) | 3.9 ± 0.7 (n = 9) | 0.09 |
| Urinary K⁺/Cr | 3.8 ± 0.4 (n = 9) | 4.5 ± 0.4 (n = 9) | <0.01 |

BW, body weight;
Cr, creatinine;
ns, not significant;
n = number of animals.

TABLE 4
Blood parameters under normal and high-calcium dietary conditions in
VDR−/− mice compared to wild-type.

| | Wild-type | VDR^{-/-} | P value |
|---|---|---|---|
| Normal diet | | | |
| Na⁺ (mmol/L) | 148.7 ± 4.9 (n = 10) | 148.3 ± 2.6 (n = 8) | ns |
| K⁺ (mmol/L) | 5.2 ± 0.9 (n = 10) | 4.8 ± 0.6 (n = 8) | ns |
| Creatinine (mg/dL) | 0.29 ± 0.1 (n = 10) | 0.24 ± 0.1 (n = 8) | ns |
| Glucose (mg/dL) | 116.2 ± 4.1 (n = 3) | 115 ± 13.3 (n = 5) | ns |
| Insulin (ng/ml) | 0.42 ± 0.2 (n = 6) | 0.38 ± 0.1 (n = 8) | ns |
| HCa-Lac diet for 5 | | | |
| weeks | | | |
| Na⁺ (mmol/L) | 143.7 ± 3.9 (n = 5) | 143.1 ± 1.7 (n = 5) | ns |
| K⁺ (mmol/L) | 4.4 ± 0.9 (n = 5) | 4.2 ± 0.3 (n = 5) | ns |
| Creatinine (mg/dL) | 0.26 ± 0.1 (n = 5) | 0.25 ± 0.1 (n = 5) | ns |

Ns, not significant;
n = number of animals.

TABLE 5
Chemical formulae for the vitamin D analogue compounds

| Compound # | Compound chemical name |
|---|---|
| 1 | 1,25-dihydroxy-16-ene-cholecalciferol |
| 2 | 1,25-dihydroxy-16-ene-23-yne-cholecalciferol |
| 3 | 1,25-dihydroxy-20-cyclopropyl-cholecalciferol |
| 4 | 1,25-dihydroxy-21(3-methyl-3-hydroxy-butyl)- |
| | cholecalciferol |
| 5 | 1,25-dihydroxy-19-nor-cholecalciferol |
| 6 | 1,25-dihydroxy-16-ene-19-nor-cholecalciferol |
| 7 | 1,25-dihydroxy-16-ene-23-yne-19-nor-cholecalciferol |
| 8 | 1,25-dihydroxy-20-cyclopropyl-19-nor-cholecalciferol |
| 9 | 1,25-dihydroxy-21(3-methyl-3-hydroxy-butyl)-19-nor- |
| | cholecalciferol |
| 10 | 1,25-dihydroxy-20R-21(3-hydroxy-3-methylbutyl)-23- |
| | yne-26,27-hexafluoro-cholecalciferol |
| 11 | 1,25-dihydroxy-20S-21(3-hydroxy-3-methylbutyl)-23- |
| | yne-26,27-hexafluoro-cholecalciferol |
| 12 | 1,25-dihydroxy-20R-21(3-hydroxy-3-methylbutyl)-23- |
| | yne-26,27-hexafluoro-19-nor-cholecalciferol |
| 13 | 1,25-dihydroxy-20S-21(3-hydroxy-3-methylbutyl)-23- |
| | yne-26,27-hexafluoro-19-nor-cholecalciferol |
| 14 | 3-Epi-1,25-dihydroxy-21(3-hydroxy-3-methylbutyl)- |
| | cholecalciferol |
| 15 | 1,25-dihydroxy-21(3-hydroxy-3-methylbutyl)-5,6-trans- |
| | cholecalciferol |
| 16 | 1-α-Fluoro-25-hydroxy-21(3-hydroxy-3-methylbutyl)- |
| | cholecalciferol |
| 17 | 1,25-dihydroxy-21(2R,3-hydroxy-3-methylbutyl)-20R- |
| | cholecalciferol |
| 18 | 1,25-dihydroxy-21(2R,3-hydroxy-3-methylbutyl)-20S- |
| | cholecalciferol |
| 19 | 1,25-dihydroxy-21(2R,3-hydroxy-3-methylbutyl)-20R- |
| | 19-nor-cholecalciferol |
| 20 | 1,25-dihydroxy-21(2R,3-hydroxy-3-methylbutyl)-20S- |
| | 19-nor-cholecalciferol |
| 21 | 1,25-dihydroxyvitamin D₃ |

### Materials and Methods

Animals and treatment. The generation and characterization of VDR^{-/-} and Gcm2^{-/-} mice have been described by Li et al. (1997) and Gunther et al. (2000). VDR^{-/-} and Gcm2^{-/-} mice were generated through breeding of heterozygous mice and identified by PCR with tail genomic DNA as the template, and the wild-type littermates were used as controls. Mice were housed in a pathogen-free barrier facility in a 12 hr light/12 hr dark cycle, and fed an autoclaved standard rodent chow. To normalize the blood ionized calcium level of VDR^{-/-} mice, two-month old animals were placed on the HCa-Lac diet (Teklad, Madison, Wis.) that contained 2% calcium, 1.25% phosphorus, 4 IU/g vitamin D, and 20% lactose for 5 weeks. To increase the sodium load, mice were fed the normal rodent diet supplemented with 8% NaCl for 1, 3, 5 and 7 days. In dehydration experiments, mice were restricted from water, but had free access to food, for 24 hr before sacrifice. To block 1,25(OH)₂D₃ synthesis, 1.5-month old wildtype mice were placed on the normal diet supplemented with 2.5% strontium chloride until hypocalcemia was detected. Wild-type mice were injected (i.p.) with vehicle or 30 pmole of 1,25(OH)₂D₃ dissolved in propylene glycol. Mice were sacrificed by exsanguination under anesthesia and the blood was collected into ice-cold tubes for serum isolation, or into ice-cold tubes containing 50 μl of EDTA (pH 8.0) and 100 U/ml aprotinin for plasma isolation. The determination of water and food intake, as well as urine collection, were carried out by using metabolic cages.

Measurement of blood and urine parameters. The concentration of blood ionized calcium was determined using a Ciba/Corning 634 Ca⁺⁺/pH analyzer (Chiron Diagnostics, East Walpole, Mass.) from 50 μl of whole blood obtained from tail snipping. Blood glucose concentrations were determined by using One Touch Sure Step test strips (Life Scan, Milpitas, Calif.). Serum intact parathyroid hormone (iPTH) was determined using a commercial ELISA kit (Immutopics, San Clemente, Calif.). The concentration of serum and urinary Na⁺, K⁺, and creatinine was determined by a Beckman Coulter CX5 Autoanalyzer as described by Li et al. (2001).

Measurement of Ang II. Mouse plasma Ang II concentrations were determined by radioimmunoassays (RIA), using a commercial RIA kit (Phoenix Pharmaceutical, Mountain View, Calif.) according to the manufacturer's instructions.

Measurement of blood pressure. Mouse blood pressure was determined as described by Liu et al. (1996). Briefly, mice were anesthetized by an intraperitoneal injection of sodium pentobarbital (50 mg/kg body weight). The left carotid artery was isolated from surrounding tissues, and cannulated with a polyethylene catheter filled with sterile phosphate-buffered saline containing heparin (50 U/ml) under a dissecting microscope. Arterial blood pressure was measured using a Harvard Apparatus pressure transducer and recorded. To investigate whether the increase in blood pressure in VDR^{-/-} mice was directly due to the increase in the Ang II level, wildtype and VDR^{-/-} mice were treated with captopril (100 mg/day/kg body weight, dissolved in drinking water) for 5 days before blood pressure was determined. Wildtype and VDR^{-/-} mice fed normal drinking water were used as controls.

Immunohistochemistry. Kidneys freshly dissected from wildtype and VDR^{-/-} mice were fixed overnight with 4% formaldehyde in PBS (pH 7.2), processed, embedded in paraffin and cut into 5-μm sections with a Leica microtone 2030. The slides were stained with a rabbit polyclonal anti-renin antiserum (1:1600 dilution) (provided by Dr. T. Inagami, Vanderbilt University). After incubation with a peroxidase-conjugated anti-rabbit IgG (KPL, Gaithersburg, Md.), the renin signal was visualized with a DAB peroxidase substrate kit (Vector Laboratories, Burlingame, Calif.), followed by a light hematoxylin counterstaining.

RNA isolation and northern blot. The kidney and liver were dissected and immediately placed into the Trizol Reagent (Invitrogen, Grand Island, N.Y.) for total RNA isolation according to the manufacturer's instruction. To determine renin or angiotensinogen mRNA expression, total RNA (20 μg/lane) was separated on a 1.2% agarose gel containing 0.6 M formaldehyde, transferred onto a Nylon membrane (MSI, Westborough, Mass.) and crosslinked in a UV crosslinker (Bio-Rad, Hercules, Calif.). Hybridization was performed as described by Li et al. (2001). Mouse renin and angiotensinogen cDNA probes were labeled with ³²P-dATP (ICN, Costa Mesa, Calif.) using the Prime-a-gene Labeling System (Promega, Madison, Wis.). After hybridization and washing, membranes were exposed to X-ray films at −80° C. for autoradiography. The relative amount of mRNA was quantitated using a PhosphoImager (Molecular Dynamics, Sunnyvale, Calif.) and normalized with 36B4 mRNA.

As4.1 cell culture and transfection. As4.1 cells (ATCC, Manassas, Va.) were cultured in DMEM supplemented with 10% FBS at 37° C. and 5% CO₂. For transient transfection, the cells were grown in 10-cm dishes to 50% confluence and transfected with pcDNA3.1, pcDNA-hVDR or pcDNA-PTH/PTHrPR plasmid (10 μg DNA/dish) by the standard calcium phosphate method. Twenty-four hr after transfection, the cells were treated for 24 hr with 5×10⁻⁸ M of 1,25(OH)₂D₃ or ethanol in serum-free media, or with different doses of bovine PTH(1-34) as indicated. Total RNA was isolated and analyzed for renin mRNA expression by northern blot. For stable transfection, As4.1 cells were transfected with pcDNA3.1 or pcDNA-hVDR plasmid by the use of Superfect reagent (Qiagen, Valencia, Calif.) and selected with 350 μg/ml of G418 for two weeks. Individual colonies were picked, expanded and selected for VDR expression. The As4.1-hVDR stable clones were treated with different doses of 1,25(OH)₂D₃ for 24 hr in serum-free media, and total RNA were analyzed by northern blot to examine renin expression.

Real-time RT-PCR. The mRNA levels of renin, AGT, and type Ia Ang II receptor (AT-1aR) in the heart were quantified by real-time RT-PCR. Briefly, first strand cDNAs were synthesized from 5 μg of total heart RNAs in a 50 μl reaction using M-MLV reverse transcriptase (Invitrogen Life Technologies) and oligo-dT12-18 as the primer. The cDNAs were then used as the template (5 μl per reaction) for real-time PCR amplification. Real-time PCR was carried out using a Cepheid Smart Cycler (Cepheid, Sunnyvale, Calif.) and a SYBR Green PCR Reagents kit (Applied Biosystems, Foster City, Calif.). The PCR primers for mouse renin, AGT, AT-1aR, and GAPDH genes were designed based on cDNA sequences deposited in GenBank database. GAPDH was used as the internal control for each reaction. All primers were tested for their specificity by conventional PCR before being used for the real-time PCR quantitative studies. The Ct value for each gene was obtained from the real-time PCR reactions, and the starting amount of each target mRNA was calculated based on a calibration curve and the Ct value. The relative amount of mRNA was normalized to GAPDH mRNA.

Therapeutic compositions: Pharmaceutical compositions used in the practice of the foregoing methods can be formulated into pharmaceutical compositions comprising a carrier suitable for the desired delivery method. Suitable carriers include materials that when combined with the therapeutic composition retain the anti-tumor function of the therapeutic composition. Examples include, but are not limited to, any of a number of standard pharmaceutical carriers such as sterile phosphate buffered saline solutions, water, and the like. Therapeutic formulations can be solubilized and administered via any route capable of delivering the therapeutic composition to the tumor site. Potentially effective routes of administration include, but are not limited to, intravenous, parenteral, intraperitoneal, intramuscular, intratumor, intradermal, intraorgan, orthotopic, and the like. A formulation for intravenous injection comprises the therapeutic composition in a solution of preserved bacteriostatic water, sterile unpreserved water, and/or diluted in polyvinylchloride or polyethylene bags containing sterile sodium chloride for injection. Therapeutic protein preparations can be lyophilized and stored as sterile powders, preferably under vacuum, and then reconstituted in water (containing for example, benzyl alcohol preservative) or in sterile water prior to injection. Dosages and administration protocols will generally depend on a number of other factors appreciated in the art.

Renin gene promoter analysis. Plasmid pR1C-4.1CAT that contains 4.1 kb 5′-flanking sequence of mouse Ren-1^{c} gene (Petrovic et al. 1996) was provided by Dr. K. W. Gross (Roswell Park Cancer Institute, Buffalo, N.Y.). To generate pGL-117 bp reporter plasmid, the 123 by renin minimal promoter fragment (+6 to −117) was released from pR1C-4.1CAT with XbaI and BamHI and inserted into the HindIII site of pGL3-basic vector (Promega, Madison, Wis.). To generate pGL-4.1 kb reporter plasmid, the BamHI fragment (−4.1 kb to −118 bp) from pR1C-4.1CAT was inserted into the BglII site of pGL-117 bp. To analyze the activity of renin gene promoter, As4.1-hVDR cells were transfected with the reporter plasmids by electroporation according to Shi et al. (2001) using a Bio-Rad Gene Pulser (Bio-Rad, Hercules, Calif.). pCMV-β-gal plasmid was co-transfected as an internal control. pGL3-control plasmid (Promega) was used as the positive control. The transfected cells were treated with ethanol or 10⁻⁸ M of 1,25(OH)₂D₃ in Opti-MEM medium (Invitrogen) containing 2% charcoal-treated FBS four hr after electroporation, and luciferase activity was determined at 48 hr after initial transfection, using the Luciferase Assay System (Promega). Luciferase activity was normalized to β-gal activity obtained from the same electroporation, and presented as fold induction based on the basal activity of pGL3-basic empty vector determined in the same experiment.

Statistical analysis. Data were presented as mean±SD and analyzed with student's t-test to assess significance. P values of 0.05 or lower were considered statistically significant.

Structural compounds. Structural formulations for Vitamin D analogues or derivatives, Gemini compounds and Gemini analogues are as described in U.S. Pat. Nos. 6,030,962; 6,559,138; 6,329,538; 6,331,642; 6,452,028; and 4,225,525, each of which is herein incorporated by reference. As disclosed herein, analogues or derivatives of Vitamin D include Gemini compounds and Gemini analogues and any other molecule that structurally resembles Vitamin D.

An exemplary Vitamin D analog or derivative has a structure that resembles:

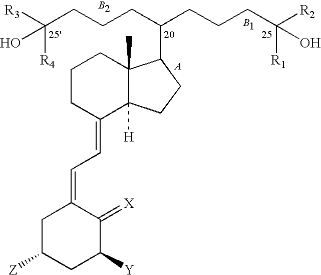

wherein:

X is H₂ or CH₂;

Y is hydrogen, hydroxy or fluorine;

Z is hydroxy;

R₁ and R₂ are a (C₁-C₄)alkyl or fluoroalkyl, or R₁ and R₂ together with C₂₅ form a (C₃-C₆)cycloalkyl or cyclofluoroalkyl;

R₃ and R₄ are a (C₁-C₄)alkyl or fluoroalkyl, or R₃ and R₄ together with C₂₅, form a (C₃-C₆)cycloalkyl or cyclofluoroalkyl;

A is a single bond or a double bond;

B₁ is a single bond, an E-double bond, a Z-double bond or a triple bond; and

B₂ is a single bond, an E-double bond, a Z-double bond or a triple bond. Data.

### DOCUMENTS

The following publications are incorporated by reference to the extent they relate to the protocols used in this disclosure.
Alroy, I., Towers, T. L., and Freedman, L. P. 1995. Transcriptional repression of the interleukin-2 gene by vitamin D3: direct inhibition of NFATp/AP-1 complex formation by a nuclear hormone receptor. Mol Cell Biol. 15:5789-5799.
Antonipillai, I., and Horton, R. 1985. Role of extra- and intracellular calcium and calmodulin in renin release from rat kidney. Endocrinology. 117:601-606.
Ballermann, B. J., Zeidel, M. L., Gunning, M. E., and Brenner, B. M. 1991. Vasoactive peptides and the kidney. In The kidney. B. M. Brenner, and F. C. Rector, editors. W.B. Saunders Company, Philadelphia. 510-583.
Broulik, P. D., Horky, K., and Pacovsky, V. 1986. Effect of parathyroid hormone on plasma renin activity in humans. Horm Metab Res. 18:490-492.
Brunner, H. R., Laragh, J. H., Baer, L., Newton, M. A., Goodwin, F. T., Krakoff, L. R., Bard, R. H., and Buhler, F. R. 1972. Essential hypertension: renin and aldosterone, heart attack and stroke. N Engl J Med. 286:441-449.
Burgess, E. D., Hawkins, R. G., and Watanabe, M. 1990. Interaction of 1,25-dihydroxyvitamin D and plasma renin activity in high renin essential hypertension. Am J Hypertens. 3:903-905.
Cheng, H. F., Wang, J. L., Zhang, M. Z., Wang, S. W., McKanna, J. A., and Harris, R. C. 2001. Genetic deletion of COX-2 prevents increased renin expression in response to ACE inhibition. Am J Physiol Renal Physiol. 280:F449-456.
Dardenne, O., Prud'homme, J., Arabian, A., Glorieux, F. H., and St-Arnaud, R. 2001. Targeted inactivation of the 25-hydroxyvitamin D(3)-1(alpha)-hydroxylase gene (CYP27B1) creates an animal model of pseudovitamin D-deficiency rickets. Endocrinology. 142:3135-3141.
Fitzsimons, J. T. 1980. Angiotensin stimulation of the central nervous system. Rev Physiol Biochem Pharmacol. 87:117-167.
Geisterfer, A. A., Peach, M. J., and Owens, G. K. 1988. Angiotensin II induces hypertrophy, not hyperplasia, of cultured rat aortic smooth muscle cells. Circ Res. 62:749-756.
Gunther, T., Chen, Z. F., Kim, J., Priemel, M., Rueger, J. M., Amling, M., Moseley, J. M., Martin, T. J., Anderson, D. J., and Karsenty, G. 2000. Genetic ablation of parathyroid glands reveals another source of parathyroid hormone. Nature. 406:199-203.
Hackenthal, E., Paul, M., Ganten, D., and Taugner, R. 1990. Morphology, physiology, and molecular biology of renin secretion. Physiol Rev. 70:1067-1116.
Imaoka, M., Morimoto, S., Kitano, S., Fukuo, F., and Ogihara, T. 1991. Calcium metabolism in elderly hypertensive patients: possible participation of exaggerated sodium, calcium and phosphate excretion. Clin Exp Pharmacol Physiol. 18:631-641.
Kimura, Y., Kawamura, M., Owada, M., Oshima, T., Murooka, M., Fujiwara, T., and Hiramori, K. 1999. Effectiveness of 1,25-dihydroxyvitamin D supplementation on blood pressure reduction in a pseudohypoparathyroidism patient with high renin activity. Intern Med. 38:31-35.
Kjaer, A., Knigge, U., Jorgensen, H., and Warberg, J. 1998. Dehydration-induced renin secretion: involvement of histaminergic neurons. Neuroendocrinology. 67:325-329.
Kong, J., Li, X. J., Gavin, D., Jiang, Y., and Li, Y. C. 2002. Target expression of human vitamin D receptor in the skin promotes the initiation of postnatal hair follicular cycle and rescue the alopecia in vitamin D receptor null mice. J. Invest. Dermatol. 118:631-638.
Krause, R., Buhring, M., Hopfenmuller, W., Holick, M. F., and Sharma, A. M. 1998. Ultraviolet B and blood pressure. Lancet. 352:709-710.
Kristal-Boneh, E., Froom, P., Harari, G., and Ribak, J. 1997. Association of calcitriol and blood pressure in normotensive men. Hypertension. 30:1289-1294.
Li, Y. C., Amling, M., Pirro, A. E., Priemel, M., Meuse, J., Baron, R., Delling, G., and Demay, M. B. 1998. Normalization of mineral ion homeostasis by dietary means prevents hyperparathyroidism, rickets, and osteomalacia, but not alopecia in vitamin D receptor-ablated mice. Endocrinology. 139:4391-4396.
Li, Y. C., Bolt, M. J. G., Cao, L.-P., and Sitrin, M. D. 2001. Effects of vitamin D receptor inactivation on the expression of calbindins and calcium metabolism. Am J Physiol Endocrinol Metab. 281:E558-E564.
Li, Y. C., Pirro, A. E., Amling, M., Delling, G., Baron, R., Bronson, R., and Demay, M. B. 1997. Targeted ablation of the vitamin D receptor: an animal model of vitamin D-dependent rickets type II with alopecia. Proc Natl Acad Sci USA. 94:9831-9835.
Lind, L., Hanni, A., Lithell, H., Hvarfner, A., Sorensen, O. H., and Ljunghall, S. 1995. Vitamin D is related to blood pressure and other cardiovascular risk factors in middle-aged men. Am J Hypertens. 8:894-901.
Lind, L., Wengle, B., Wide, L., and Ljunghall, S. 1989. Reduction of blood pressure during long-term treatment with active vitamin D (alphacalcidol) is dependent on plasma renin activity and calcium status. A double-blind, placebo-controlled study. Am J Hypertens. 2:20-25.
Liu, S. Q., and Fung, Y. C. 1996. Indicial functions of arterial remodeling in response to locally altered blood pressure. Am J Physiol. 270:H1323-1333.
Nishishita, T., Okazaki, T., Ishikawa, T., Igarashi, T., Hata, K., Ogata, E., and Fujita, T. 1998. A negative vitamin D response DNA element in the human parathyroid hormone-related peptide gene binds to vitamin D receptor along with Ku antigen to mediate negative gene regulation by vitamin D. J Biol Chem. 273:10901-10907.
Omdahl, J. L., and DeLuca, H. F. 1971. Strontium induced rickets: metabolic basis. Science. 174:949-951.
Pan, L., Black, T. A., Shi, Q., Jones, C. A., Petrovic, N., Loudon, J., Kane, C., Sigmund, C. D., and Gross, K. W. 2001. Critical roles of a cyclic AMP responsive element and an E-box in regulation of mouse renin gene expression. J Biol Chem. 276:45530-45538.
Pan, L., Xie, Y., Black, T. A., Jones, C. A., Pruitt, S. C., and Gross, K. W. 2001. An Abd-B class HOX.PBX recognition sequence is required for expression from the mouse Ren-1c gene. J Biol Chem. 276:32489-32494.
Panda, D. K., Miao, D., Tremblay, M. L., Sirois, J., Farookhi, R., Hendy, G. N., and Goltzman, D. 2001. Targeted ablation of the 25-hydroxyvitamin D 1alpha-hydroxylase enzyme: evidence for skeletal, reproductive, and immune dysfunction. Proc Natl Acad Sci USA. 98:7498-7503.
Park, C. W., Oh, Y. S., Shin, Y. S., Kim, C. M., Kim, Y. S., Kim, S. Y., Choi, E. J., Chang, Y. S., and Bang, B. K. 1999. Intravenous calcitriol regresses myocardial hypertrophy in hemodialysis patients with secondary hyperparathyroidism. Am J Kidney Dis. 33:73-81.
Petrovic, N., Black, T. A., Fabian, J. R., Kane, C., Jones, C. A., Loudon, J. A., Abonia, J. P., Sigmund, C. D., and Gross, K. W. 1996. Role of proximal promoter elements in regulation of renin gene transcription. J Biol Chem. 271:22499-22505.
Pfeifer, M., Begerow, B., Minne, H. W., Nachtigall, D., and Hansen, C. 2001. Effects of a short-term vitamin D(3) and calcium supplementation on blood pressure and parathyroid hormone levels in elderly women. J Clin Endocrinol Metab. 86:1633-1637.
Polly, P., Herdick, M., Moehren, U., Baniahmad, A., Heinzel, T., and Carlberg, C. 2000. VDR-Alien: a novel, DNA-selective vitamin D(3) receptor-corepressor partnership. Faseb J. 14:1455-1463.
Resnick, L. M., Muller, F. B., and Laragh, J.H.1986. Calcium-regulating hormones in essential hypertension. Relation to plasma renin activity and sodium metabolism. Ann Intern Med. 105:649-654.
Ritthaler, T., Scholz, H., Ackermann, M., Riegger, G., Kurtz, A., and Kramer, B. K. 1995. Effects of endothelins on renin secretion from isolated mouse renal juxtaglomerular cells. Am J Physiol. 268:F39-45.
Rostand, S. G. 1997. Ultraviolet light may contribute to geographic and racial blood pressure differences. Hypertension. 30:150-156.
Shi, Q., Gross, K. W., and Sigmund, C. D. 2001. Retinoic acid-mediated activation of the mouse renin enhancer. J Biol Chem. 276:3597-3603.
Sigmund, C. D., Okuyama, K., Ingelfinger, J., Jones, C. A., Mullins, J. J., Kane, C., Kim, U., Wu, C. Z., Kenny, L., Rustum, Y., et al. 1990. Isolation and characterization of renin-expressing cell lines from transgenic mice containing a renin-promoter viral oncogene fusion construct. J Biol Chem. 265:19916-19922.
Silver, J., Naveh-many, T., Mayer, H., Schmeizer, H. J., and Popovtzer, M. M. 1986. Regulation by vitamin D metabolites of parathyroid hormone gene transcription in vivo in the rat. J. Clin. Invest. 78:1296-1301.
Skott, O., and Briggs, J. P. 1987. Direct demonstration of macula densa-mediated renin secretion. Science. 237:1618-1620.
Smith, J. M., Mouw, D. R., and Vander, A. J. 1983. Effect of parathyroid hormone on renin secretion. Proc Soc Exp Biol Med. 172:482-487.
Tamura, K., Chen, Y. E., Horiuchi, M., Chen, Q., Daviet, L., Yang, Z., Lopez-Ilasaca, M., Mu, H., Pratt, R. E., and Dzau, V. J. 2000. LXRalpha functions as a cAMP-responsive transcriptional regulator of gene expression. Proc Natl Acad Sci USA. 97:8513-8518.
Towers, T. L., and Freedman, L. P. 1998. Granulocyte-macrophage colony-stimulating factor gene transcription is directly repressed by the vitamin D3 receptor. Implications for allosteric influences on nuclear receptor structure and function by a DNA element. J Biol Chem. 273:10338-10348.
Yoshizawa, T., Handa, Y., Uematsu, Y., Takeda, S., Sekine, K., Yoshihara, Y., Kawakami, T., Arioka, K., Sato, H., Uchiyama, Y., et al. 1997. Mice lacking the vitamin D receptor exhibit impaired bone formation, uterine hypoplasia and growth retardation after weaning. Nature Genetics. 16:391-396.
U.S. Pat. No. 6,030,962
U.S. Pat. No. 6,559,138
U.S. Pat. No. 6,329,538
U.S. Pat. No. 6,331,642
U.S. Pat. No. 6,452,028
U.S. Pat. No. 4,225,525

We claim:

1. A method of reducing blood pressure in a mammal in need thereof, by suppressing renin expression, the method comprising:
(a) obtaining a pharmaceutical composition comprising vitamin D or a vitamin D analogue or derivative of formulae
(i) 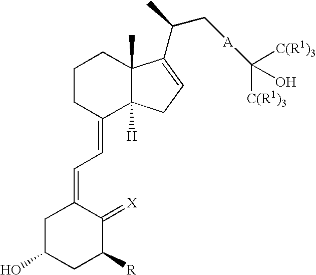
where R¹ is in each instance independently selected from hydrogen or halogen; R is hydrogen and X is ═CH₂, or R is hydroxy and X is H₂ or ═CH₂; and
A is —C≡C—, 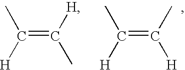
or —CH₂—CH₂—, provided that when A is —CH₂—CH₂—, R¹ is hydrogen;
(ii) 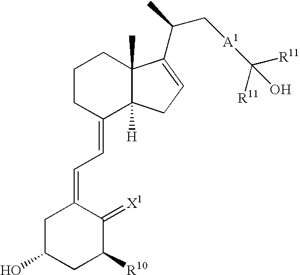
where each R¹¹ is independently selected from the group consisting of ethyl, propyl, butyl, isopropyl and t-butyl; R¹⁰ is hydrogen and X is ═CH₂, or R¹⁰ is hydroxy and X is H₂ or ═CH₂; and
A¹ is —C≡C—, 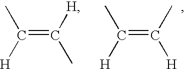
or —CH₂—CH₂—;
(iii) 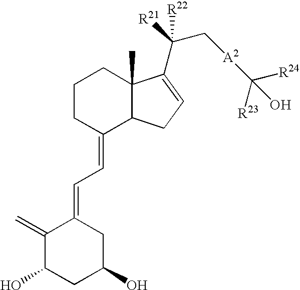
where R²¹ and R²² are each independently hydrogen or alkyl, or R²¹ and R²² and the attached carbon form cyclopropyl; R²³ and R²⁴ are each independently selected from the group consisting of alkyl, hydroxyalkyl and fluoroalkyl; and
A² is —C≡C—, 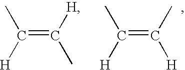
or —CH₂—CH₂—;
(iv) 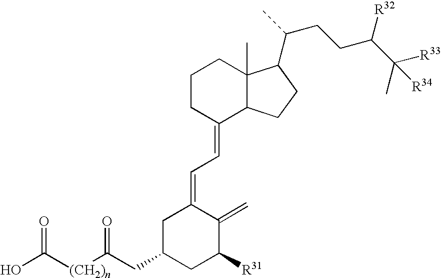
where R³¹, R³², R³³, and R³⁴ are each independently selected from the group consisting of hydrogen, hydroxy and fluoro; provided that at least one of R³¹, R³², R³³, or R³⁴ is hydroxy; and n is 2 or 3;
(v) 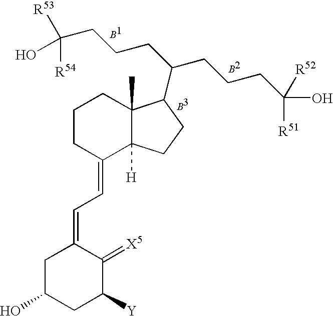
where X⁵ is H₂ or ═CH₂; Y is hydrogen, hydroxy or fluoro; R⁵¹ and R⁵² are each independently C₁-C₄ alkyl or fluoroalkyl, or R⁵¹ and R⁵² together with the attached carbon form a C₃-C₆ cycloalkyl or cyclofluoroalkyl; R⁵³ and R⁵⁴ are each independently C₁-C₄ alkyl or fluoroalkyl, or R⁵³ and R⁵⁴ together with the attached carbon form a C₃-C₆ cycloalkyl or cyclofluoroalkyl; B¹ and B² are each independently selected from the group consisting of a single bond, an E-double bond, a Z-double bond or a triple bond; and B³ is a single or a double bond;
(vi) 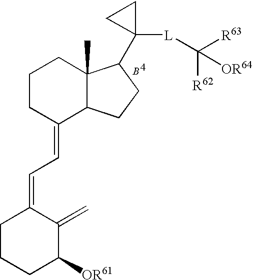
where B⁴ is a single or a double bond; R⁶¹ and R⁶⁴ are each independently selected from the group consisting of hydrogen, alkyl, acyl, and a hydroxy protecting group, provided that at least one of R⁶¹ and R⁶⁴ is acyl; R⁶² and R⁶³ are each independently alkyl or haloalkyl, or R⁶² and R⁶³ together with the attached carbon form a cycloalkyl; and L is selected from the group consisting of —CH₂—CH₂—CH₂—, —CH₂—CH═CH—, —CH₂—C≡C—, —CH₂—CH₂—C(O)—, and —CH═CH—CH═CH—;
or a pharmaceutically acceptable salt thereof; and
(b) administering the pharmaceutical composition to the mammal.

2. The method of claim 1, wherein the mammal is a human.

3. The method of claim 1, wherein the risk of stroke, myocardial infarction, congestive heart failure, cardiac hypertrophy, progressive atherosclerosis and renal failure is reduced.

4. The method of claim 1, wherein the pharmaceutical composition is a sustained release formulation.

5. The method of claim 1, wherein the pharmaceutical composition comprises an angiotensin-converting enzyme inhibitor.

6. The method of claim 1, wherein the pharmaceutical composition comprises an angiotensin II type I receptor antagonist.

7. The method of claim 1, wherein the vitamin D is a hormonal form of vitamin D designated as 1,25-dihydroxyvitamin D3.
